(19) United States
(12) Patent Application Publication
(10) Pub. No.: US 2004/0038894 A1
(43) Pub. Date: Feb. 26, 2004

(54) Compounds for modulating cell negative regulations and biological applications thereof

(75) Inventors: Marc Daeron, Chevreuse (FR); Eric Vivier, Cassis (FR)

Correspondence Address: NIXON & VANDERHYE, PC, 1100 N GLEBE ROAD, 8TH FLOOR, ARLINGTON, VA, 22201-4714, US

(73) Assignee: INSTITUT NATIONAL DE LA SANTE ET DE LA RECHERCHE MEDICALE (I.N.S.E.R.M.)

(21) Appl. No.: 10/615,773

(22) Filed: Jul. 10, 2003

Related U.S. Application Data
(63) Continuation of application No. 09/331,885, filed on Sep. 2, 1999, now abandoned.
Application No. 09/331,885 is a 371 of international application No. PCT/IB97/01610, filed on Dec. 31, 1997.

(30) Foreign Application Priority Data
Dec. 31, 1996 (GB) 96 402 930.0

(51) Int. Cl. A61K 38/17; C07K 14/705

(52) U.S. Cl. 514/12; 530/350

(57) ABSTRACT

The invention relates to compounds for modulating cell negative regulations and biological applications thereof. It particularly relates to compounds capable of cross-linking a simulatory receptor with a KIR (Killer-cell inhibitory receptor).

The invention relates to compounds capable of modulating cell negative regulations. It also relates to the biological applications of said compounds. Cell negative regulations dysfunctions can lead to diseases such as allergic, inflammatory or cytotoxicity-related diseases.

Cytotoxicity is a major strategy used by the immune system to eliminate cellular antigens such as virus-infected cells and tumor cells.

NK (Natural Killer cells) cells are spontaneously cytotoxic lymphocytes, capable of recognizing antigens expressed by tumoral cells.

NK cells are also involved in autoimmune, immunoproliferative and immunodeficiency diseases.

NK cells can induce the lysis of target cells by two mechanisms. Antibody-dependent cell cytotoxicity (ADCC) leads to the lysis of antibody-coated target cells, whereas natural cytotoxicity leads to the antibody-independent lysis of a variety of cell targets, including primarily virus-infected cells and tumor cells.

NK cells represent a peculiar class of lymphocytes which cannot rearrange antigen receptor gene segments. NK cells are however capable of recognizing and inducing the lysis of deleterious cells, and primarily in vitro tumor cells as well as virus-infected cells.

A major mechanism which controls NK cell cytotoxic function is initiated by the recognition of MHC Class I molecules expressed at the surface of target cells. NK cells express several cell surface receptors for MHC Class I molecules, i.e. the so called KIR (Killer-cell Inhibitory Receptors).

In contrast to the T cell receptor complex (CD3/TCR), KIR are characterized (i) by their ability to interact with a large panel of MHC Class I allele products (promiscuous recognition), and (ii) by their ability to transduce a negative signal which leads to the inhibition of both natural cytotoxicity and ADCC programs.

KIR are not NK cell-restricted since they are also expressed on T cell subsets and can inhibit T cell activation triggered via the CD3/TCR complexes.

Human and mouse KIR (MHC Class I inhibitory receptors) belong to two distinct families: immunoglobulin superfamily (IgSF) and C2 lectins. Lectin-like KIR are receptors for MHC Class I molecules and also for carbohydrates. In particular, human KIR IgSF include CD158 (p58), Cdw159 (p70) and Cdw160 (p140) molecules whereas human KIR C2 lectin include the CD94-NKG2A/B heterodimer.

KIR are therefore all expressed on NK cells but none of them are NK-restricted. They can therefore not only be involved in autoimmune but also in inflammatory diseases and immunoproliferative aid immunodeficiency diseases. Research relating to cell regulation has up to now focused on cell receptors for Fc (immunoglobulin constant fragments) such as FcγRIIB, and on antigen-specific receptors of T and B cells.

The balance between receptor-mediated activation and inactivation is central to in vivo homeostasis.

The cell surface receptors initiating NK cell activatory pathways comprise:

i. the ADCC receptor complex, including FcγRIIIA (CD16, which is the only Fc receptor expressed on NK cells), KAR (Killer cell Activatory Receptor) and a variety of disulfide-linked hetero- and homodimers associated with CD16. The engagement of the ADCC receptor initiates a series of ITAM-dependent (Immunoreceptor Tyrosine-based Activation Motifs) signaling pathways, leading to the release of intracytoplasmic NK granules as well as the transcription of a set of genes encoding surface activation molecules (e.g. CD69, CD25) and cytokines (e.g. α-IFN),

ii. the NK receptors mediating activatory signals for the initiation of natural cytotoxicity programs, such as NKRP-1 proteins,

iii. Lag 3, a molecule expressed on activated T and NK cells which is homologous to CD4.

iv. adhesion molecules, such as the Beta-2 integrin expressed on NK cells or DNAM-1 expressed in most T and NK lymphocytes and on a subpopulation of B lymphocytes.

From the prior art teaching, four mechanisms can be essentially considered as modulating cell activation. These mechanisms could lead to

a direct interference with the ligand-activatory receptor binding events, such has been observed in cytokine biology with the interleukin-1 receptor antagonist,

a down-regulation of the activatory receptor membrane expression, such has been observed with the epidermal growth factor receptor,

an interference with the effector function coupled to the activation receptor, i.e. an interference with the transcription of the set of genes induced by the activation cascade, such has been observed with the glucocorticoids, or

an interference with the early signaling pathway coupled to the activation receptor, such has been observed with the heterotrimeric G-protein or with the receptors coupled to PTK activation.

The present invention herein demonstrates that a KIR, i.e. a SHP-1/SHP-2 recruiting ITIM-bearing receptor, necessarily require co-aggregation with activatory receptors to exert their inhibitory functions on said activatory receptors.

The present invention also demonstrates that KIR, normally expressed on NK or T cells, can function in non-lymphoid cells, and that KIR can thereby inhibit the activation of receptors involved in inflammatory and allergic responses.

The present invention therefore surprisingly demonstrates that a KIR is capable of modulating the activation of ITAM-bearing receptors.

The present invention also gives the first report of the obtention of a specific anti-ITIM (Immunoreceptor Tyrosine-based Inhibition Motif) compound.

The present invention further demonstrates that the KIR family exerts regulatory functions and uses strategies to mediate its inhibitory functions distinct and divergent from those exerted and used by other members of the ITIM-bearing receptor family. The present invention in particular gives the first demonstration that in contrast to other ITIM-bearing receptors, a KIR, which is an ITIM-bearing receptor that does not recruit SHIP but that does recruit SHP-1 and/or SHP-2, is capable of inhibiting the release of Ca²⁺ from intracellular stores upon co-aggregation with an ITAM-bearing receptor. It also gives the first demonstration that in contrast to other ITIM-bearing receptors, the co-aggregation of a KIR and of an ITIM-bearing receptor greatly enhances the tyrosine phosphorylation of KIR ITIMs, but that it is not mandatory to KIR phosphorylation. The present invention also gives for the first time the demonstration that a KIR, and a human KIR in particular, in vivo control the host tolerance to allogeneic grafts such as bone marrow or skin grafts.

One aspect of the invention accordingly relates to a compound capable of cross-linking a stimulatory receptor with a KIR.

In many embodiments, it will be desirable to provide a compound capable of specifically regulating the activation of a KIR and/or capable of regulating the activation of a stimulatory receptor.

Said stimulatory receptor is particularly an ITAM-bearing receptor such as KAR, FcεRI, CD3/TCR, CD16, any receptor related to tyrosine kinase activities, such as a growth factor receptor, or a receptor sub-unit such as CD3ζ, CD3ε, CD3γ, CD3δ or FcεRIγ.

Said KIR is an IgSF member, such as CD158 (p58), CDw159 (p70), CDw160 (p140), or is lectin-like, such as the CD94/NKG2A heterodimer. Said KIR is advantageously a human KIR.

Said KIR may be expressed on a NK, a T or a mast cell or on a monocyte or is recombinantly expressed.

The compound of the invention is further characterized in that it is capable of inducing the regulation of free calcium concentration in a cell. Said compound is most preferably capable of inducing the regulation of calcium influx into a cell and/or of calcium mobilization from intracellular compartments.

Said compound is further characterized in that it is capable of inducing the recruitment by said KIR of SH2-domain containing protein tyrosine phosphatases, and particularly of a phosphatase selected from the group consisting of SHP-1, SHP-2.

In preferred embodiments, said compound is essentially a polypeptide, a glycoprotein or a carbohydrate.

In other preferred embodiments, said compound is a bispecific reagent and/or a chemical inducer of dimerization. It may be produced by chemical synthesis or by genetic engineering.

In yet other preferred embodiments, said compound is a bispecific antibody. For example, said compound may comprise at least one Fab, Fd, Fv, dAb, CDR, F(ab′)₂, VH, VL, ScFv fragment.

In most preferred embodiments, said compound is capable of cross-linking said KIR with said stimulatory receptor in the extracellular domain of a cell. Said compound can advantageously cross-link a stimulatory receptor with any KIR that is Ig-like or with any KIR that is lectin-like.

In other most preferred embodiments, said compound is capable of crossing through a lipid bi-layer. For example, it may be liposoluble and/or associated with a drug-delivery system.

In yet other most preferred embodiments, said compound is capable of cross-linking said KIR with said stimulatory receptor in the intracellular domain of a cell. Said compound can advantageously cross-link a stimulatory receptor with definite KIR (Ig-like or lectin-like) or indiscriminately with any KIR (Ig-like and lectin-like). Said compound may be advantageously associated with a drug-delivery system.

In certain preferred embodiments, said compound is capable of modulating the release of serotonin and/or of inflammatory mediators by a cell expressing FcεRI, such as a mast cell, and/or of modulating cytokine release (Interleukin-6, Tumor Necrosis Factor Alpha release) from a cell, such as a mast cell or a NK cell, and/or of modulating interleukin production such as the IL-2 production and/or the γ-interferon production from a peripheral blood cell and/or of modulating the proliferation of peripheral blood cells.

In another most preferred embodiment, said compound is capable of controling the host tolerance to allogeneic grafts such as bone marrow grafts or skin grafts and/or the graft toxicity against host tissues (Graft Versus Host) against host tissues. Such a compound is thus capable of preventing the development of an immune response mounted against the cells of the host, or against the cells of the graft.

Another aspect of the invention provides a nucleic acid coding for a polypeptide according to the invention and a cell transfected by said nucleic acid.

In still another aspect, the invention relates to a pharmaceutically acceptable preparation comprising a therapeutically-effective amount of at least one compound at the invention. Such a pharmaceutical preparation is useful for modulating an animal cell function involved in a disease selected from the group consisting of immunoproliferative diseases, immunodeficiency diseases, cancers, autoimmune diseases, infectious diseases, viral diseases, inflammatory responses, allergic responses or involved in organ transplant tolerance.

The pharmaceutical preparation of the invention may be formulated in solid or liquid form or in suspension for oral administration, parenteral administration, topical, intravaginal or intrarectal application, or for nasal and/or oral inhalation.

The present invention also makes available a method for the in vitro or ex vivo diagnosis of a cell disregulation, comprising the step of estimating of the relative proportion of co-aggregated KIR vs. non-co-aggregated, KIR by:

contacting a biological sample with a compound, or with a nucleic acid, or a cell according to the invention, and

of revealing the reaction product possibly formed.

Estimating the relative proportion of co-aggregated KIR vs non-co-aggregated KIR is particularly useful for the precise diagnosis of diseases where cell disregulation is involved, such as immunoproliferative diseases, immunodeficiency diseases, cancers, autoimmune diseases, infectious diseases, viral diseases, inflammatory responses, allergic responses and for the choice of the appropriate treatment.

Other aspects and embodiments of the present invention will become obvious to one of ordinary skill in the art after consideration of the drawing and examples provided below. What follows should not be interpreted as limiting the invention in any way.

### SEVENTEEN FIGURES ARE MENTIONED

FIG. 1 illustrates the reconstitution of wild-type and mutant p58.2 HLA-Cw3-specific KIR in RTIIB cells,

FIG. 2 illustrates the surface receptor-induced serotonin release in RTIIB cells expressing human KIRs,

FIG. 3 shows that human KIRs inhibit ITAM-dependent RTIIB cells serotonin release,

FIG. 4 shows that the inhibition of ITAM-dependent RTIIB cells serotonin release requires KIR co-aggregation,

FIG. 5 shows that human KIRS inhibit ITAM-dependent intracytoplasmic Ca²⁺ mobilization in RTIIB cells,

FIG. 6 shows immunofluorescence and flow cytometry analysis of peripheral blood lymphocytes isolated from p58.2 transgenic mice,

FIGS. 7(A, B) illustrates the in vitro cytotoxicity of splenic T cells isolated from CD158b (p58.2) transgenic mice, and

FIG. 8 shows a schematic representation of the CD158b (p58.2) transgenic vector used for generation of transgenic mice,

FIG. 9 illustrates the in vitro cytotoxicity of splenic NK cells isolated from CD158b (p58.2) transgenic mice (Tg CD158b) and from nontransgenic littermate (non Tg), and

FIG. 10 illustrates the tolerance of CD158b (p58.2) transgenic mice to craft of allogeneic bone marrow cells that express HLA-Cw3(mean cpm±SEM of incorporated ¹²⁵IdUdr obtained from three independent grafts)?

FIG. 11 illustrates that NKG2A and CD94 are expressed on NK cells and melanoma specific T-cell clones,

FIGS. 12(A, B) illustrates that (CD94-NKG2A engagement inhibits cytotoxicity on NKL cells and melanoma specific T-cell clones,

FIG. 13 illustrates that CD94-NKG2A inhibits the antigen-specific TNF production by CTL clones,

FIG. 14 illustrates the negative regulation of antigen-induced CTL clone cytotoxicity by CD94-NKG2A,

FIGS. 15 (A, B) illustrates the in vitro interaction between NKG2A ITIMs and SHP-1, SHP-2 and SHIP phosphatases,

FIG. 16 illustrates the BIAcore analysis of NKG2A ITIM interaction with the SH2 domains of SHP-1, SHP-2 and SHIP phosphatases (top: NKG2AN-term phosphorylated ITIM; bottom: NKG2AC-term phosphorylated ITIM), and

FIGS. 17 (A, B) illustrates the in vivo recruitment of SHP-1 and SHP-2 by phosphorylated NKG2A.

ABBREVIATIONS

| | |
|---|---|
| ADCCR | Antibody-Dependent Cell Cytotoxicity Receptor |
| | complex. |
| Ca²⁺: | Intracellular Ca²⁺ concentration. |
| DAM: | Donkey Anti-Mouse Ig antiserum. |
| DAR: | Donkey Anti-Rat Ig antiserum. |
| FITC: | Fluorescin isothiocyanate. |
| GAM: | Goat Anti-Mouse Ig antiserum. |
| GST: | Glutathion S-transterase. |
| IgSF: | Immunoglobulin superfamily. |
| ITAM: | Immunoreceptor Tyrosine-based Activation Motif. |
| ITIM: | Immonoreceptor Tyrosine-based Inhibition Motif. |
| KAR: | Killer-cell Activatory Receptor. |
| KIR: | Killer-cell Inhibitory Receptor. |
| Kd: | Equilibrium dissociation constant |
| mAb: | monoclonal Antibody. |
| MHC: | Major Histocompatibility Complex. |
| NK: | Natural Killer. |
| PBL: | Peripheral Blood Lymphocytes. |
| PTK: | Protein Tyrosine Kinase. |
| PTPase: | Protein Tyrosine Phosphatase. |
| SHIP: | Phosphatidylinositol phosphatase |
| SH2: | src-homology domain 2. |
| SPR: | Surface plasmon resonance. |
| TC: | Tricolor. |
| Tg: | transgenic |

### EXAMPLE 1

KIR can function in a non lymphoid cell line,

KIR only inhibit the functions of the activatory receptors with which they are co-aggregated (serotonin release, Ca²⁺ influx and mobilization),

KIR and Fc ITIM-bearing receptors exert distinct regulatory functions. HLA-Cw3-specific KIR (p58.2) has been reconstituted in a transfected mast cell/basophil-like RHL-2H3 cell line: (RTIIB). RTIIB cells express two distinct ITAM-dependent receptors: the endogenous FcεRI antigen receptor and a transfected CD25/CD3ζ chimeric molecule. A naturally-occuring mutant of p58.2, the p50.2 KAR (Killer-cell Activatory Receptor), has also been reconstituted in RTIIB cells. The p50.2 KAR expresses a shorter intracytoplasmic domain, which does not contain any I/VxYxxL/V stretch (ITIM motif). This mutant receptor is able to trigger T and NK cell activation programs.

Experimental Procedures:

Antibodies. The following mAb and antisera were obtained from Immunotech, Marseille, France: mouse anti-human CD25 mAb (B1.49.9, IgG2a), rat anti-human CD25 mAb (33 B3.1, IgG2a), mouse anti-human p58.2 mAb (GL183, IgG1), mouse anti-rat Ig (H+L) F(ab′)₂(MAR), goat anti-mouse Ig (H+L) F(a,b)₂ (GAM), donkey anti-mouse Ig (H+L) F(ab′)₂ (DAM), donkey anti-rat Ig (H+L) F(ab′)₂(DAR). Rat IgE mAb (LO-DNP-30), mouse IgE mAb (2682-I), rat anti-FcγRII/III mAb (2.4G2), mouse anti-CD25 mAb (7G7,IgG1) and mouse anti-rat FcεRIα (BC4,IgG1) were also used. Mouse IgE mAb was used as a dilution of hybridoma supernatants. All other mAb were used as protein A/G purified mAb. GL183, 2.4G2 and 7G7 mAb were used as F(ab′)₂; LO-DNP-30, 2682-I, B1.49.9 and 33.B3.1 were used as intact mAb. LO-DNP-30 and 2682-I are directed against DNP and TNP. MAR F(ab′)₂ was trinitrophenylated using trinitrobenzene sulfonic acid (Eastman-Kodak, Rochester, N.Y., USA), 1 mole of MAR F(ab′)₂ was substituted with an average number of 10 TNP moles. This TNP-F(ab′)₂MAR was used to crosslink mouse anti-TNP IgE and rat anti-FcγRII/III 2.4G2.

Cells. All cells were cultured in DMEM supplemented with 10% FCS and penicillin (100 IU/ml)-streptomycin (100 μg/ml). RHL-2H3 cell transfectants expressing murine FcγRIIb2 and CD25/CD3ζ chimeric molecules (RTIIB) have been previously described. The CD25/CD3ζ chimeric molecule includes the complete human CD25 ecto- and transmembrane domains linked to the complete mouse CD3ζ intracytoplasmic domain. RTIIB cells were transfected by electroporation using either the 183.6 cDNA encoding p58.2 (RTIIBp58) or the 183.Act1 cDNA encoding p50.2 (RTIIBp50), in the RSV-5.gpt expression vector. Stable transfectants were established by culture in the presence of xanthine (250 μg/ml), hypoxanthine (13.6 μg/ml) and mycophenolic acid (2 μg/ml). Two representative clones of each transfection series (RTIIBp58A, RTIIBp58B and RTIIBp50A, RTIIBp50B) were examined in parallel and gave similar results. Unless indicated, results from one clone of each transfection series are shown.

Flow cytometric analysis. The primary mAb was incubated with cells on ice for 20-30 minutes, followed by 3 washes with PBS supplemented with 0.2% BSA. The secondary staining was performed using fluorescein-conjugated rabbit anti-mouse IgG (Immunotech, Marseille, France), followed by 3 P BS/0.2% BSA washes and resuspension in PBS containing 1% formaldehyde. Cells were analyzed on a FACS-Scan apparatus (Becton-Dickinson, Mountain View, Calif., USA).

Single cell Ca²⁺ video-imaging. Cells (2×10⁵/sample) were allowed to adhere overnight onto glass coverslips in culture medium. Adherent cells were washed and then incubated at 37° C. for 40 minutes in RPMI medium supplemented with 10% FCS with a 10⁻³ dilution of mouse IgE (2682-I) in the presence or absence of either 1 μg/ml GL183 or 1 μg/ml 2.4G2. 1 μM Fura-2/AM (Molecular Probes, Eugene, Oreg. USA) in dimethylsulfoxide premixed with 0.2 mg/ml pluronic acid (Molecular Probes) was then added to the medium for 20 minutes. Cells were washed and then measurements of intracytoplasmic Ca²⁺ (Ca²⁺₁) mobilization were performed at 37° C. in MS buffer (140 mM NaCl, 5 mM KCl, 10 mM HEPES, pH 7.4, 1 mM CaCl₂, 1 mM MgCl₂) with a Nikon Diaphot 300 microscope and an IMSTAR imaging system. Briefly, each [Ca²⁺]₁ image (taken every 6 seconds) was calculated from the ratio of the average of 4 fluorescence images after 340 nm excitation, and 4 fluorescence images after 380 nm excitation. Ca²⁺₁ values were calculated according to Grynkiewicz et al. 1985, J. Biol. Chem. 260: 3440-3450. The stimulation was done by adding GAM F(ab′)₂ or TNP-F(ab′)₂MAR to the MS buffer to a final concentration of 50 μg/ml and 10 μg/ml respectively. The measurements of intracellular calcium stocks release was done by replacing the MS buffer with a stimulation buffer (150 mM NaCl, 5 mM KCl, 1 mM MgCl2, 0.5 mM EGTA, 20 mM HEPES, pH 7.3, 50 μg/ml F(ab′)₂ GAM) at the time of stimulation. For each experiments, results were obtained as the average variation of Ca²⁺₁ (nM) as a function of time for a population of 20-30 cells.

Serotonin release. RTIIB cell transfectants harvested using trypsin-EDTA were examined for serotonin release. Briefly, cells were resuspended in RPMI medium supplemented with 10% FCS at 1×10⁶ cells/ml, and incubated at 37° C. for 1 h with 2 μCi/ml [³H] serotonin (Amersham, Les Ulis, France), washed, resuspended in RPMI-10% FCS, incubated for another hour at 37° C., washed again, resuspended in the same medium and distributed in 96-well microculture plates at 2×10⁵ cells/well. Cells were then incubated for 1 h with mouse or rat IgE, with mouse or rat anti-CD25 mAb in the absence or in the presence of GL183 F(ab′)₂, in a final volume of 50 μl. Cells were washed 3 times in 200 ul P BS and once in 200 μl RPMI-10% FCS, then 25 μl culture medium were added to each well and cells were warmed for 15 minutes at 37° C. before challenge. Cells were challenged for 30 minutes at 37° C. with 25 μl prewarmed GAM, DAM or DAR F(ab′)₂ as indicated. Reactions were stopped by adding 50 μl ice-cold medium and by placing plates on ice. 50 μl of supernatants were mixed with 1 ml Emulsifier Safe scintillation liquid (Packard, Groningen, The Netherlands) and counted in a LS6000 Beckman counter. The percentage of serotonin release was calculated using as 100%, cpm contained in 50 μl harvested from wells containing the same number of cells and lysed in 100 μl 0,5% SDS-0.5% NP40.

Results

Reconstitution of human KIR and KAR in RTIIB cells. Stable RTIIB cell transfectants expressing murine FcγRIIB as well as a CD25/CD3ζ chimeric molecule at their surface, were further transfected with two distinct NK cell MHC Class I receptor p58.2 and p50.2 cDNA constructions. In NK cells, the wild type p58.2 (KIR) exerts an inhibitory effect whereas the mutant p50.2 (KAR) is an activating molecule. Despite this striking difference, both p58.2 and p50.2 are HLA-Cw3-specific receptors and are recognized by the GL183 mAb. Representative transfected clones used thereafter were selected for their matched cell surface expression of the wild-type p58.2 (RTIIBp58) or the mutant p50.2 (RTIIBp50) HLA-Cw3 receptors.

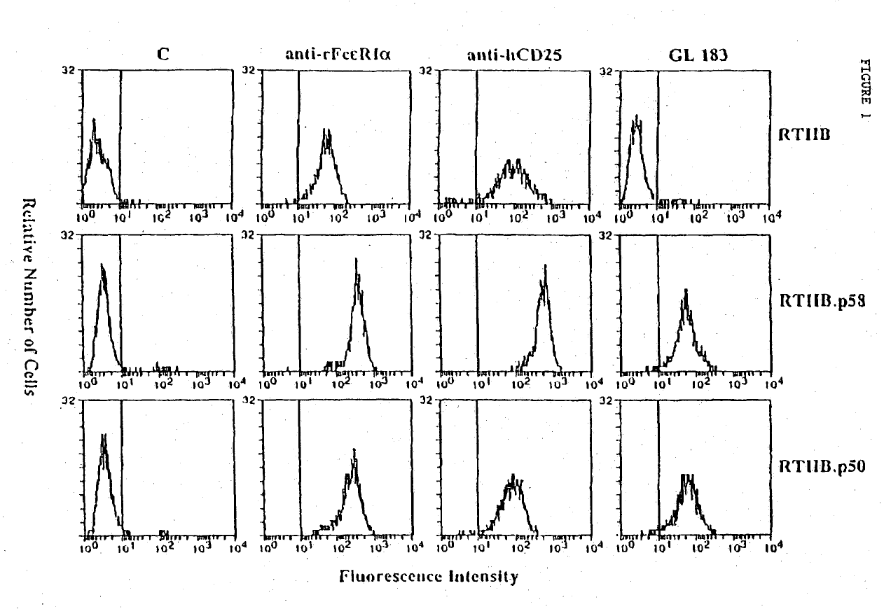

FIG. 1 illustrates the reconstitution of wild-type and mutant p58.2 HLA-Cw3-specific KIR in RTIIB cells, where representative transfected RTIIB clones were stained by indirect immunofluorescence using mAb directed against rat Fce RI (BC4), human CD25 (B1.49.9), as well as human wild-type p58.2 KIR and mutant p50.2 KAR (GL183). Negative controls were only incubated with fluorescein conjugated rabbit anti-mouse IgG also used as secondary reagents.

Inhibition of ITAM-dependent serotonin release by KIR reconstituted in RTIIB cells. RTIIB cells can be induced to release serotonin by one of two ways: aggregation of the endogenous rat FcεRI receptor complex or aggregation of the CD25/CD3ζ chimeric molecule. Mouse IgE binding to FcεRI is not sufficient to induce RTIIB serotonin release, and aggregation of FcεRI receptors was obtained using GAM F(ab′)₂. The integrity of ITAM expressed by both receptors is required for RTIIB serotonin release, indicating that both FcεRI- and CD25/CD3ζ-induced serotonin release utilize ITAM-dependent signaling mechanism. Using RTIIB cells expressing p58.2 KIR or the mutant p50.2 KAR in addition to FcεRI and CD25/CD3ζ, the expression or the aggregation of reconstituted HLA-Cw3 receptors, was investigated with respect to ITAM-dependent serotonin release modulation.

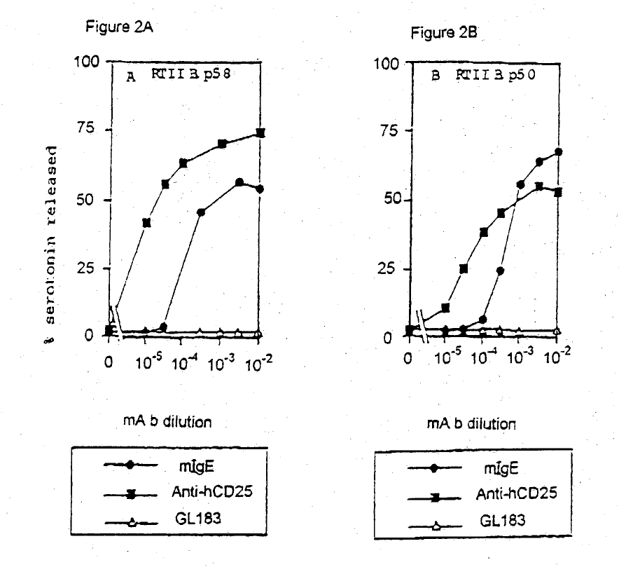

FIG. 2 illustrates the surface receptor-induced serotonin release in RTIIB cells expressing human KIR RTIIBp58 cells (A) or RTIIBp50 cells (B) were incubated 1 h at 37° C. either with serial dilution of mouse IgE (2682-I, initial concentration: straight hybridoma supernatant) (closed circles), anti-hCD25 (F(ab′)₂ 7G7, initial concentration: 1 mg/ml) (closed squares) or GL183 F(ab′)₂ (initial concentration: 1 mg/ml) (open triangles). After being washed, cells were challenged for 30 minutes at 37° C. with 50 μg/ml GAM F(ab′)₂. The serotonin released in supernatants was measured. The experiment shown is representative of three independent experiments.

As shown in FIGS. 2A and B, aggregation of FcεRI or CD25/CD3ζ receptors induces a dose-dependent serotonin release of RTIIB cells expressing either p58.2 KIR (RTIIBp58) or p50.2 KAR (RTIIBp50). The larger serotonin release elicited by anti-CD25 in RTIIBp58 compared to RTIIBp50 cells most likely reflects the difference in surface expression of CD25/CD3ζ in the two cell types. When p58.2 KIR were aggregated using anti- p58.2 mAb (GL183), no RTIIB serotonin release was observed. This result is consistent with the lack of detectable signals induced in NK and T cells upon anti-p58.2 mAb stimulation. However, no serotonin release was detected in response to GL183 mAb in RTIIBp50 cells, in contrast with the stimulatory effect of p50.2 KAR reported in both NK and T cells.

In a second set of experiments, p58.2 KIR vs. p50.2 KAR and FcεRI receptors were co-aggregated using mouse IgE, mouse GL183 and GAM.

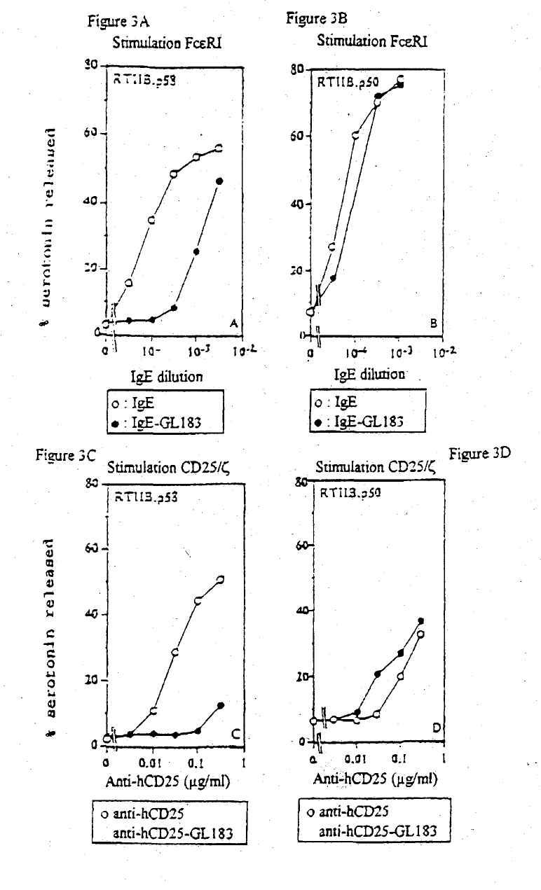

Results are reported in FIG. 3: RTIIBp58 cells (A,C) and RTIIBp50 cells (B,D) were incubated 1 h at 37° C. with 3 μg/ml GL183 F(ab′)₂ and mouse IgE (2682-I) (A and B, closed circles) or with 3 μg/ml GL183 F(ab′)₂ and anti-hCD25 (F(ab′)₂ 7G7) (C and D, closed circles). Controls were made without GL183 F(ab′)₂ (open squares). After being washed, cells were challenged for 30 minutes at 37° C. with 50 μg/ml GAM F(ab′)₂. The serotonin released in supernatants was measured. The experiment shown is representative of three independent experiments.

Using saturating concentrations of GL183, the serotonin release induced by FCεRI was impaired in RTIIBp58 cells (FIG. 3A). This inhibition was detectable at sub-optimal (<10⁻³ dilution), as well as optimal concentration of IgE (10⁻³ dilution). Using saturating concentrations of both GL183 (1 μg/ml) and IgE (10⁻³ dilution), 72.8%±2.2 and 55.8%±9.9 (mean±1 SEM, n=3) inhibition of serotonin release were observed in two distinct RTIIBp58 clones. Similar results were obtained when serotonin release was triggered via the CD25/CD3ζ chimeric molecule (FIG. 3C). Using saturating concentrations of both GL183 (1 μg/ml) and anti-hCD25 (3 μg/ml), serotonin release was inhibited by 39.3%±11.9 in the representative RTIIBp58 B clone.

In contrast, no significant inhibition or potentiation of ITAM-dependent cell activation was detected when p50.2 KAR was co-aggregated with either FcεRI or CD25/CD3ζ surface receptors, even at sub-optimal concentration of triggering IgE or anti-hCD25, in the presence of saturating concentration of GL183 (1 μg/ml) (FIGS. 3B and 3D).

These results first demonstrate that p58.2 KIR reconstituted in RTIIB cells are functional, and inhibit ITAM-dependent cell activation. Second, they show that the integrity of p58.2 intracytoplasmic sequence is required for KIR-mediated inhibition of RTIIB serotonin release. Finally, these data indicate that RTIIB cells provide an appropriate cellular environment for a functional reconstitution of p58.2 KIR, but not of p50.2, KAR.

KIR inhibitory function requires co-engagement of KIR and ITAM-containing receptors. In a next set of experiments, RTIIBp58 cells were stimulated via FcεRI in the presence of aggregated p58.2 in one of two ways.

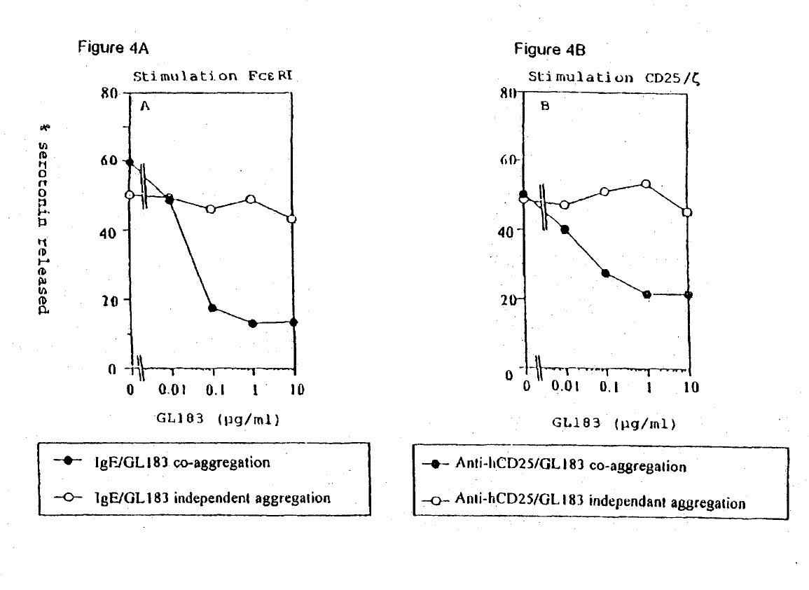

Results are reported in FIG. 4:

(A) RTIIBp58 cells were incubated 1 h at 37° C. with indicated concentrations of GL183 F(ab′) and either 10⁻³ mIgE (2682-I) dilution (closed circles) or 10⁻³rIgE (LO-DNP-30,1 mg/ml initial concentration) dilution (open circles). After being washed, cells were challenged for 30 minutes at 37° C. with 50 μg/ml DAM F(ab′): (closed circles) or with 50 μg/ml DAM F(ab′)₂ plus 50 μg/ml DAR F(ab′) (open circles). The serotonin released in supernatants was measured.

(B) RTIIBp58 cells were incubated 1 h at 37° C. with indicated concentrations of GL183 F(ab′)₂ and either 3 μg/ml anti-hCD25 mAb (mEL.49.9) (closed circles) or 3 μg/ml anti-hCD25 mAb (r33 B3.1) (open circles). After being washed, cells were challenged for 30 minutes at 37° C. with 50 μg/ml DAM F(ab′)₂ (closed circles) or with 50 μg/ml DAM F(ab′)₂ plus 50 μg/ml DAR F(ab′)₂ (open circles). The serotonin released in supernatants was measured. This experiment is representative of five independent experiments. Co-aggregation and independent aggregation experiments are schematized in (C) and (D) respectively.

DAM was used to co-aggregate FcεRI and p58.2 KIR via mouse IgE and mouse GL183 respectively (FIG. 4C), or a combination of DAR and DAM was used to independently aggregate FcεRI and p58.2 KIR via rat IgE and mouse GL183 respectively (FIG. 4D). As shown in FIG. 4A (closed circles), FcεRI-p58.2 KIR co-aggregation induced a GL183 dose-dependent inhibition of FcεRI-induced serotonin release, consistent with the observations reported in FIG. 3A. By contrast, the independent aggregation of FcεRI and p58.2 KIR failed to inhibit FcεRI-induced serotonin release at any GL183 concentration (FIG. 4A, open circles). Similar results were obtained when RTIIBp58 serotonin release was induced via the CD25/CD3ζ chimeric molecule (FIG. 4B). These results demonstrate that KIR require a co-aggregation with activatory receptors (FcεRI or CD25/CD3ζ), in order to exert their inhibitory function. They also suggest that KIR-mediated inhibition takes place in the immediate vicinity of KIR molecules. Consistent with this conclusion, co-aggregation of FcεRI and KIR only inhibits RTIIB cell serotonin release induced by FcεRI but not by triggering of the CD25/CD3ζ chimeric molecule.

An inhibitory effect of p58.2 KIR should therefore only be observed when the relative proportion of co-aggregated KIR-activatory receptors is high enough.

To test this point, a clone expressing an unusually high level of CD25/CD3ζ chimeric molecules was selected, In this particular clone, using saturating concentration of GL183 F(ab′)₂ 1 μg/ml), only low levels of inhibition (14.1%±5.0) of CD25/CD3ζ-induced serotonin release were observed.

In addition, it has been observed that in all clones stimulated with supra-optimal IgE concentrations (>10⁻³), KIR fail to inhibit FcεRI-induced RTIIB serotonin release. These data confirm that an inhibitory effect requires the co-aggregation of activatory and inhibitory receptors.

KIR-FcεRI co-aggregation inhibits FcεRI-induced intracytoplasmic Ca²⁺ mobilization. RTIIB serotonin release mediated via ITAM triggers Ca²⁺₁ mobilization. In order to test whether reconstituted human KIR inhibit early ITAM-dependent activatory signals, measurements of Ca²⁺₁ were performed using a single cell imaging system.

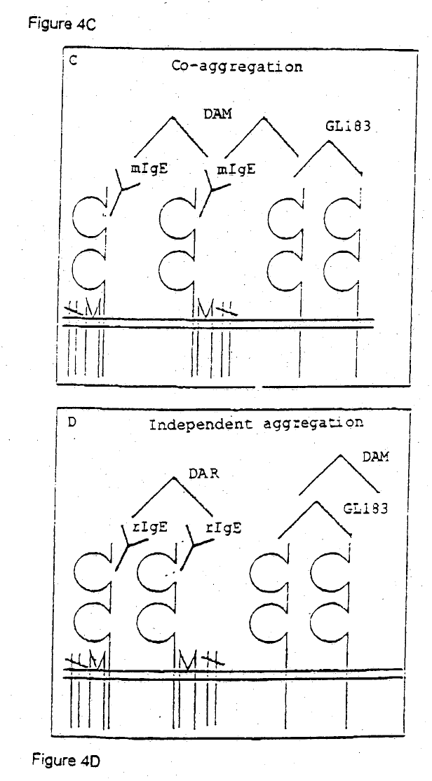

Results are reported in FIG. 5: Dotted line: RTIIBp58 cells were pre-incubated with mouse IgE mAb (2682-I) (10⁻³ dilution). Continuous lines: RTIIBp58 cells were pre-incubated 1 hour with a combination of mouse IgE mAb (10⁻³ dilution) and GL183 F(ab′)₂ mouse mAb (1 μg/ml), (A,C) or mouse IgE mAb (10⁻³ dilution) and 2.4G2 F (ab′)₂ rat mAb (1 μg/ml) (B,D). At a time indicated by the arrow, cells were stimulated with a GAM F(ab′)₂ (50 μg/ml) (A,C), or TNP-F(ab′)₂MAR (10 μg/ml) (B,D). (A,B): RTIIBp58 cells were stimulated in the presence of extracellular calcium. (C,D) RTIIBp58 cells were stimulated in the absence of extracellular calcium. Values were obtained from 59 to 117 tested cell from 3 to 5 independent experiments.

RTIIBp58 and RTIIBp50 cells were stimulated via the FcεRI receptor complex in the absence or presence of GL183 using GAM as a cross-linker. In both RTIIBp58 and RTIIBp50 cells, aggregation of the FcεRI receptor complex induced a large Ca²⁺₁ response consisting in a peak followed by a sustained plateau (FIG. 5A, dotted line). When p58.2 KIR was co-aggregated with FcεRI, the IgE-induced Ca²⁺₁ peak was impaired (FIG. 5A, continuous line). Using saturating concentrations of both GL183 F(ab′)₂ (1 μg/ml) and IgE (10⁻³ dilution), the Ca²⁺₁ reached at the peak decreased from 1459 nM±92 (n=58 cells) to 756 nM±59 (n=60 cells) (mean±SEM, p<0.001) (see Table 1 below).

TABLE 1
Control of Ca²⁺, mobilization In RTIIB, RTIIB.p50 and
RTIIB.p58 cells

| | IgE | IgE + | IgE | IgE + 2.4G2 |
|---|---|---|---|---|
| | + | GL183 + | + | + |
| | GAM | GAM | TNP-F(ab¹)₂MAR | TNP-F(ab¹)₂MAR |
| RTIIB | | | | |
| Basal Level^{(a)} | 104 ±5 | 104 ± 3 | | |
| Peak response^{(b)} | 1234 ± 78 | 1287 ± 68 | N.D.^{(f)} | N.D.^{(f)} |
| Plateau^{(c)} | 645 ± 35 | 692 ± 36 | | |
| RTIIB.p50 | | | | |
| Basal Level^{(a)} | 145 ± 5 | 165 ± 6 | | |
| Peak response^{(b)} | 829 ± 67 | 873 ± 67 | N.D.^{(f)} | N.D.^{(f)} |
| Plateau^{(c)} | 519 ± 26 | 534 ± 25 | | |
| RTIIB.p58 | | | | |
| Basal Level^{(a)} | 110 ± 4 | 100 ± 5 | 102 ± 5 | 96 ± 4 |
| Peak response^{(b)} | 1459 ± 92 | 756 ± 59 | 1032 ± 47 | 985 ± 48 |
| Plateau^{(c)} | 700 ± 36 | 339 ± 25 | 515 ± 27 | 253 ± 12 |
| RTIIB.p68 | | | | |
| without calcium^{(e)} | | | | |
| Basal Level^{(a)} | 99 ± 3 | 118 ± 5 | 104 ± 7 | 79 ± 2 |
| Peak response^{(b)} | 625 ± 39 | 368 ± 32 | 712 ± 32 | 901 ± 36 |
| Plateau^{(c)} | 67 ± 3 | 94 ± 6 | 74 ± 4 | 66 ± 3 |

IgE (2682-1), GL183 F(ab¹)₂, 2.4G2 F(ab¹)₂, GAM F(ab¹)₂, and TNP-MAR F(ab¹)₂ were used as indicated in FIG. 5.
^{(a)}(Ca²⁺)₁ at 2 minutes, before stimulation in nM ± SEM.
^{(b)}(Ca²⁺)₁ at the peak of the response in nM ± SEM.
^{(c)}(Ca²⁺)₁ at 17 minutes in nM ± SEM.
^{(d)}(Ca²⁺)₁ at 12 minutes in nM ± SEM.
^{(e)}the experiment was performed in the absence of extracellular calcium and in the presence of 0.5 mM EGTA as indicated in experimental procedures.
^{(f)}N.D.: Not done.
Values were obtained from 59 to 117 tested cells from 3 to 5 independent experiments.

In contrast, when p50.2 KAR was co-aggregated with FcεRI, no significant alteration of IgE-induced Ca²⁺₁ mobilization was observed. At the Ca²₁ peak, using saturating concentrations of both GL183 (1 μg/ml) and IgE (10⁻³ dilution), Ca²⁺₁ was 829 nM±67 (n=67 cells) vs. 873 nM±67 (n=67 cells) (mean±SEM) for IgE vs. IgE-GL183 co-aggregation respectively (Table 1).

In order to evaluate whether KIR inhibited ITAM-dependent release of Ca²⁺₁ from intracellular stores and/or Ca²⁺₁ influx, further experiments were performed on RTIIBp58 cells in the absence of extracellular Ca²⁺. In these conditions, only a small peak corresponding to the release of Ca²⁺ from intracellular stores was observed upon IgE stimulation (FIG. 5C, dotted line). Using saturating concentrations of both GL183 (1 μg/ml) and IgE (10⁻³ dilution), Ca²⁺₁ decreased from 625 nM±39 (n=56 cells) to 368 nM±32 (n=37 cells) (mean±SEM, p<0.001) at the Ca²⁺₁ peak (see Table 1 above).

Therefore, p58.2 KIR is able to inhibit the release of Ca²⁺ from intracellular stores upon co-aggregation with FcεRI. In addition, no Ca²⁺₁ mobilization was detected when p58.2 or p50.2 were aggregated on RTIIBp58 and RTIIBp50 cells in the absence of IgE stimulation.

These results show that p58.2 KIR impairs ITAM-induced Ca²⁺ mobilization in RTIIB cells. Furthermore, p50.2 KAR was not capable of mediating any detectable Ca²⁺ mobilisation when expressed in RTIIB cells, in contrast to its stimulatory function reported in both T and NK cells. FcγRIIB-FcεRI co-aggregation inhibits extracellular Ca²⁺ influx in RTIIB cells, but does not inhibit intracellular Ca²⁺ mobilization.

RTIIBp58 cells also express FcγRIIB ITIM-bearing receptor. Similar to KIR, FcγRIIB inhibit serotonin release in RTIIB cells. But by contrast to KIR, FcγRIIB only inhibit Ca²⁺ entry in B cells, Therefore, the effect of FcγRIIB-FcεRI co-aggregation on Ca²⁺ mobilization was examined, in order to state whether the differential effect of KIR and FcγRIIB on Ca²⁺ mobilization is due to a difference between B cells and RTIIB cells or is the consequence of distinct inhibitory strategies used by these two ITIM-bearing receptors.

RTIIBp58 cells pre-incubated with IgE (10⁻³ dilution) in the presence or absence of 2.4G2 F(ab′)₂ (1 μg/ml), were challenged with a TNP-F(ab′)₂MAR (10 μg/ml). Aggregation of the FcεRI receptor complex induced a large Ca²⁺₁ response consisting in a peak followed by a sustained plateau (FIG. 5B, dotted line). When FcγRIlB was co-aggregated with FcεRI, the Ca²⁺₁ peak was not impaired but the plateau was not sustained (FIG. 5B, continuous line). Using saturating concentrations of both 2.4G2 F(ab′)₂ (1 μg/ml) and IgE (10⁻³ dilution), the Ca²⁺₁ reached at the plateau decreased from 515 nM±27 (n=60 cells) to 253 nM±12 (n=59 cells) (mean±SEM, p<0.001) (see Table 1 above).

In order to dissect the effects of FcγRIIB inhibition on Ca²⁺ mobilization, further experiments were performed on RTIIBp58 cells in the absence of extracellular Ca²⁺. In these conditions, only a peak corresponding to the release of Ca²⁺ from intracellular stores was observed upon IgE stimulation (FIG. 5D, dotted line). Using saturating concentrations of both 2.4G2 (1 μg/ml) and IgE (10⁻³ dilution), no inhibition of Ca²⁺ release was observed, but rather Ca²⁺₁ increased from 712 nM±32 (n=79 cells) to 901 nM±36 (n=59 cells) (mean±SEM) at the Ca²⁺₁ peak (Table 1 above and FIG. 5D). These results indicate that FcγRIIB has no effect on the release of Ca²⁺ from intracellular stores upon co-aggregation with FcεRI. In addition, no Ca²⁺₁ mobilization was detected when FcγRIIB was aggregated on RTIIBp58 cells in the absence of IgE stimulation.

Therefore p58.2 KIR and FcγRIIB ITIM-bearing receptors exert distinct regulatory function on FcεRI-dependent Ca²⁺ mobilization.

Discussion

HLA-Cw3-specific human KIR expressed in RTIIB cells can therefore inhibit Ca²⁺₁ mobilization and serotonin release induced by the FcεRI receptor as well as by a CD25/CD3ζ chimeric molecule (FIG. 3, FIG. 5). Therefore, KIR and FcγRIIB share several features.

First, KIR and FcγRIIB inhibit early steps of the signaling cascade, which are transcription-independent and are likely to reflect NK cell killing mechanisms, such as regulated exocytosis.

Second, KIR and FcγRIIB control the signals induced via polypeptides including only one ITAM (FcεRI), as well as receptors including three sequential ITAM (CD3ζ).

However KIR and FcγRIIB appear to use distinct inhibitory strategies. Indeed KIR inhibits Ca²⁺₁ release from ER stores whereas FcγRIIB only inhibit influx from the extracellular compartment. In addition, upon phosphorylation of the ITIM, FcγRIIB recruits preferentially the phosphatidyl inositide 5-phosphatase SHIP, whereas KIR recruits the SHP-1 tyrosine phosphatase (see Example 2). These two findings could be related.

These results also provide new insights on the importance of the cellular environment for the functions of a novel receptor family, characterized either by intact intracytoplasmic ITIM (FcγRIIB and KIR), or by their mutated version (KAR).

Finally, it is herein demonstrated that the mere cell surface expression of KIR does not modulate RTIIB cell activation in contrast to other regulators of lymphocyte activation, such as CD45.

On the contrary, co-aggregation between an activatory receptor (the FcεRI receptor complex or the CD25/CD3ζ chimeric molecule) and p58.2 KIR is required for the inhibition of serotonin release by RTIIB cells (FIG. 4). This implies that the KIR inhibitory effect occurs in the immediate vicinity of the molecule.

The absolute requirement for a co-engagement of KIR with an activatory receptor may reflect the necessity for KIR to be phosphorylated by a non diffusible PTK associated with the activatory receptor. Indeed, the tyrosine phosphorylation of KIR is mandatory to the recruitment of SHP-1. Thus, the same PTK might induce the tyrosine phosphorylation of both ITAM and ITIM, which is consistent with data showing that ITIM YxxL/V sequence is a potential substrate for the src-family member PTK used by ITAM-containing receptor, such as lyn, lck an fyn. An additional basis for the obligation of proximity between activatory and inhibitory molecules may be that the tyrosine phosphorylated targets of SHP-1 likely belong to the ITAM-dependent activation cascade, and must be thus brought in close proximity to SHP-1.

The identification of the major targets for SHP-1 involved in the KIR inhibitory pathways is not yet achieved. Nevertheless, the recognition of target cells protected from NK cell lysis by the surface expression or HLA-B alleles leads to an inhibition of phosphatidyl inositol 4,5 biphosphate hydrolysis, resulting in the prevention of Ca²⁺₁ mobilization in NK cells. Similarly, it is herein demonstrated that KIR inhibit ITAM-induced Ca²⁺₁ mobilization in RTIIB cells. Therefore, phospholipase C-γ, and/or its potential upstream signaling effector/adaptors, such as p36-38, as well as the ITAM-binding SH2-tandem PTK (ZAP-70 and p72^{Syk}) may be SHP-1 targets involved in KIR signaling pathways.

In conclusion, the present invention allows to define and extend the family of ITIM-bearing receptors, involved in the negative control of cell activation: the T cell-specific CD3/TCR complex pathway and the KIR pathway, both regulating T and NK cell cytotoxicity, are complementary, and permit to eliminate a cell presenting a foreign antigen in the context of MHC Class I molecules as well as a cell expressing no (or a modified form of) MHC Class I molecules.

T lymphocytes, the activation of which KIR can control, are involved in the control of potential autoimmune reactions as well as other inflammatory/immune reactions which may be deleterious. In addition, expression of KIR during viral infection, may overcome a deficient CD3/TCR triggering.

Indeed various virus interfere with assembly and transport of Class I molecules to the cell surface, which might result in a less efficient presentation and/or expression of structurally abnormal Class I molecules. As a result, CTL will be less efficiently stimulated by the CD3/TCR complexes, but more efficiently stimulated because of the absence of KIR engagement by MHC Class I molecules.

The above-reported findings therefore demonstrate that the threshold of T cell activation depends not only on the TCR-ligand avidity and the number of TCR engaged, but also depends on the engagement or the non-engagement of KIR. Several alternative pathways are utilized in T cells to control the activation programs, and similarly to KIR these pathways appear to act on the early PTK-dependent steps of T cell activation.

### EXAMPLE 2

In contrast to FcγRIIB, KIR do not bind SHIP and recruit SHP-1 and SHP-2.

Obtention of an antiserum specifically recognizing the tyrosine phosphorylated but not the non-phosphorylated form of both the N- and C-terminal KIR ITIMs.

Experimental procedures:

Peptides and antisera. The following peptides were synthesized as phosphorylated or not, and contain an N-terminal-biotin:

| PEPTIDE | SEQUENCE |
|---|---|
| p58.2.1 (N-terminal | DEQDPQEVTY₃₀₃AQLNH |
| ITIM): | |
| p58.2.1-V³⁰¹A: | DEQDPQEATY₃₀₃AQLN |
| p58.2.2 (C-terminal | RP SQ RPKTPPTDIIVY₃₃₃TELPNAEP |
| ITIM) | |
| FcγRIIB: | KTEAENTITY₂₆₂SLLK |
| FcγRIIB-I₂₆₀A: | KTEAENTATY₂₆₂SLLK |
| CD3ξ1: | YQGQNQLY₇₁ NELNLGRREEY₈₂DVLDKRRGR |

The p58.2 peptides correspond to the human p58. 2 KIR sequence. The FcγRIIB peptides correspond to the murine FcγRIIB1/B2 sequence which is highly homologous to the human sequence: KVGAENTITYSLLM. The murine sequence was chosen because of the impossibility at generating phosphopeptides corresponding to the human sequence. The 712 rabbit antiserum was generated using phosphorylated p58.2.1 peptide coupled to ovalbumine as an immunogen (Neosystem).

Fusion proteins and surface plasmon resonance analysis. Surface plasmon resonance (SPR) measurements were performed on a BIAcore™ (Pharmacia). The GST-SHP-1.SH2(NC), GST-SHP-1.SH2(N), GST-SHP-1.SH2(C), GST-SHIP.SH2 fusion proteins generated from the murine phosphatase cDNA, were purified from DH5α lysates. Briefly, overnight culture at 37° C. in LB medium containing 50 μg/ml ampicillin was diluted 1/10 in fresh medium, and incubated until the absorbance at 600 nm reaches 1-2. At that point, IPTG was added (1 mM), and incubation continued for an additional 4 hours at 37° C. After centrifugation, bacteria pellet was resuspended in TENGN buffer (50 mM Tris pH 8, 1 mM EDTA, 10% glycerol, 1% NP40, 1 mM DTT, 1 mM PMSF, 10 μg/ml aprotinin and 10 μg/ml leupeptine). Bacteria were lysed by sonication. After centrifugation, supernatant was incubated with Glutathione-Sepharose 4B beads (Pharmacia) overnight at 4° C. with slow shaking. After 3 washes with 50 mM Tris pH 8, elution of the fusion protein was carried out using 50 mM Tris pH 8 supplemented with 10 mM reduced glutathione. Before their use in BIAcore™ experiments, fusion proteins were dialyzed in HBS buffer (10 mM HEPES, 150 mM NaCl, 3.4 mM EDTA). Protein purity was assessed by 12.5% SDS-PAGE, and Coomassie blue staining. The running buffer used in all BIAcore™ experiments consisted of HBS buffer supplemented with 0.05% surfactant P20. For preparation of microchips coated with phosphorylated peptides, immunopure streptavidine (Sigma) was first immobilized onto CMS-sensorchip. Then, biotinylated peptides were injected, and their binding to streptavidine-coated microchips was assessed using anti-phosphotyrosine 4G10 mAb (UHI).

Assay for cell lysate adsorption to peptides. RBL-2H3 cells were lysed in NP-40 lysis buffer (1% NP-40, 10 mM Tris-HCl, 150 mM NaCl, 1 mM EDTA, 1 mM PMSF, 10 mM iodoacetamide, 10 mM NaF, 10 mM Na pyrophosphate, 0.4 mM Na vanadate, 10 μg/ml leupeptine, 10 μg/ml aprotinine). Samples were either used directly (whole cell lysates: WCL), or subjected to affinity purification using peptides bound to beads. Biotinylated peptides (5 μg) were coupled to 50 μl streptavidin-agarose bead slurry (Sigma) for 1 hour at 4° C., prior to bead saturation with D-biotin (1 mg/ml) for 1 hour at 4° C. After 3 washes in lysis buffer, samples were separated on SDS-PAGE and transferred to nitrocellulose. Immunoblots were revealed using anti-SHP-1, anti-SHP-2 mAb (0.5 μg/ml) (Transduction laboratories) or anti-SHIP antiserum and ECL (Amerzham).

Cell activation and immunoblotting.

Cells from a representative clone of each transfected cell line, were washed 3 times in cold PBS, resuspended at 3×10⁶ cells/ml in cold PBS and incubated for 30 min at 4° C. in the presence or absence of the indicated purified mAb (5 μg/ml). After 1 wash in PBS, cells were resuspended at the same concentration in the presence of 5 μg/ml F(ab′)₂ goat anti-mouse antiserum (GAM, Immunotech) for 3 min at 37° C. Cells were then instantly lysed in NP-40 lysis buffer for 15 min on ice. After removing insoluble material by centrifugation at 12,000 rpm for 15 min, samples were either used directly or subjected to immunoprecipitation for 2 hr using GL183 covalently coupled to Cn Br-beads (Pharmacia). Samples were then combined with reducing sample buffer (New England Biolabs) and boiled, before separation on 10% SDS-polyacrylamide gel electrophoresis (SDS-PAGE). Immunoblots using 712 were revealed using horseradish peroxydase-conjugated goat anti-rabbit antiserum (Sigma) and ECL detection system (Amersham).

Results and discussion:

In order to dissect the signaling pathways used by KIR, we first investigated whether the co-aggregation between the HLA-Cw3-specific p58.2 KIR, and an ITAM-bearing receptor, FcεRI, could modulate the tyrosine phosphorylation of KIR ITIM in vivo. In this regard, antisera directed towards the tyrosine phosphorylated form of KIR ITIM peptides were generated. Among this series, antiserum 712 specifically recognizes the tyrosine phosphorylated-, but not the non-phosphorylated form of both the N- and the C-terminal KIR ITIMs. The 712 antiserum was further used to probe whole cell lysates as well as anti-KIR immunoprecipitations prepared from stimulated and unstimulated reconstituted KIR cells. RBL-2H3 cell transfectants which express endogenous FcεRI and exogenous p58.2 KIR (RTIIB p58 cells), were stimulated using anti-FcεRI (BC4), anti-p58.2 KIR (GL183) mAbs alone or in combination, in the presence of GAM as a cross-linker. Although tyrosine phosphorylation of KIR was detected in whole cell lysates upon anti-p58.2 KIR cross-linking, a major increase in KIR tyrosine phosphorylation was observed when p58.2 KIR was co-aggregated with the FCεRI ITAM-containing complex. KIR contain only 2 intracytoplasmic tyrosine residues which are included in the N- and C-terminal ITIMs respectively.

Therefore, reactivity of the 712 anti-KIR ITIM antiserum can only account for KIR ITIM tyrosine phosphorylation. These results were confirmed using 712 immunoblotting of anti-p58.2 immunopre-cipitations. As a control, no tyrosine phosphorylation of KIR was detected when cell lysates were prepared from the RTIIBp50 cell transfectants. RTIIB50 cells express p50.2, a naturally-occurring mutant of p58.2, that contains a truncated form of p58.2 intracytoplasmic domain which is devoid of both ITIMs and inhibitory function. These results indicate that co-aggregation between KIRs and ITAM-bearing receptors greatly enhances the tyrosine phosphorylation of KIR ITIMs, consistent with its requirement for KIR inhibitory function. However, co-aggregation of KIR with FcεRI is not mandatory to KIR tyrosine phosphorylation, in agreement with the reported association of KIRs with the p56^{lck} PTK. Therefore, co-aggregation between KIRs and ITAM-bearing receptors may be also required at a later step than KIR tyrosine phosphorylation. Indeed, phosphorylated forms of KIR ITIM recruit SHP-1 and SHP-2 PTPases suggesting that co-aggregation is required for bringing the PTPases in the close vicinity of their tyrosine phosphorylated substrates, which likely belong to the ITAM-dependent cascade. In this regard, the SH2-tandem PTK, ZAP-70 has been recently shown to be a direct substrate of SHP-1.

In contrast to KIR, FcγRIIB cross-linking in RBL-2H3 cell transfectants does not lead to any detectable FcγRIIB ITIM tyrosine phosphorylation, suggesting that these two distinct ITIM-bearing negative coreceptors use diverse strategies in order to mediate their inhibitory function.

Since the inositol polyphosphate 5-phosphate, SHIP and the phosphatases SHP-1, SHP-2 are involved in FcγRIIB inhibitory function, the ability of both KIR N- and C-terminal ITIMs to bind SHIP in vitro was investigated.

Whereas both phosphorylated forms of KIR ITIM bind SHP-1 and SHP-2, no binding to SHIP was detected. As a control, only the tyrosine phosphorylated form of FcγRIIB ITIM recruit SHIP, SHP-1 and SHP-2 phosphatases. The recombinant GST-fusion protein and SPR analysis confirmed that the phosphorylated form of KIR ITIM does not associate with SHIP, in contrast to FcγRIIB ITIM. The absence of direct SHIP binding in vitro to KIR ITIM is consistent with the absence of SHIP recruitment to KIR in vivo, and is likely to have physiological implications. Indeed we have previously observed (see example 1) that in the same RTIIBp58 cells, which express both FcγRIIB and p58.2 KIR, the mechanisms used by both ITIM-bearing receptors to inhibit FcεRI-induced cell activation are divergent: whereas KIR and FcεRI co-aggregation leads to inhibition of Ca²⁺ release from the endoplasmic reticulum, FcγRIIB and FcεRI co-aggregation leads to the inhibition of Ca²⁺ influx form the extracellular milieu.

The differential recruitment of PTPases or SHIP by KIR and FcγRIIB may therefore be involved in the differential effect of both ITIM-bearing negative coreceptors on Ca²⁺ mobilization. In this regard, SHIP is a polyphosphate inositol 5-phosphatase which remains to be characterized for its function as a regulator of phosphatidyl-inosotol 4,5 biphosphate and inositol 1,4,5 triphosphate metabolism. Irrelevant of their differential binding properties, KIR and FcγRIIB ITIM share a common isoleucine or valine residues at a position tyrosine-2. Similarly, another hematopoietic ITIM-bearing negative coreceptor gp49 BL, which is expressed on mast cells and inhibits FcεRI-mediated activation, also contains isoleucine and valine residues at the position tyrosine-2 in its two ITIMs respectively.

Phosphorylated KIR and FcγRIIB ITIM peptides corresponding to a single point aminoacid substitution, i.e. p58.2.1-V₃₀₁A and FcγRIIB 1-I₂₆₀A, were generated. These peptides were tested for their ability to bind SHP-1, SHP-2, and SHIP phosphatases in a cell lysate adsorption assay, in comparison to the wild type KIR and FcγRIIB ITIM peptides. Substitution of isoleucine and valine by an alanine residue abolishes the binding of SHP-1 and SHP-2 to both KIR and FcγRIIB ITIM peptides.

In contrast, binding of SHIP to mutated FcγRIIB ITIM is not affected. Similar results were obtained using SPR for SHP-1 and SHIP, confirming that the tyrosine-2 amino-acid position in ITIMs is critical for binding to PTPase but not to SHIP. Since KIRs do not associate with SHIP, we also further characterized the interaction between KIRs and SHP-1, which is likely to be involved in the inhibitory function of KIRs. We thus determined using SPR the kinetic constants of the interaction between KIR ITIM peptides and recombinant fusion proteins corresponding to the isolated N- and C-SH2 domains of SHP-1. Both the isolated SHP-1. SH2 domains bind the phosphorylated form of KIR ITIMs, in contrast to previous results reporting that only the C-SH2 domain was responsible of the interaction between KIP and SHP-1.

In the following table 2, are shown the association and dissociation constants for the interaction of isolated SHP-1.SH2(N) and SHP-1.SH2(C) domains with phosphorylated KIR ITIM peptides:

TABLE 2

| Phospho- | GST-SHP-1, SH2 (N) | | | GST-SHP-1, SH2 (C) | | |
|---|---|---|---|---|---|---|
| rylated | kₒₙ | k_{off} | K_{d} | kₒₙ | k_{off} | Kⱼ |
| peptides | (10⁻⁴M⁻¹s⁻¹) | (10³ s⁻¹) | (nM) | (10⁻⁴M⁻¹s⁻¹) | (10³ s⁻¹) | (nM) |
| p58.2.1 | 10.0 | 42 | 423 | 0.50 | 0.7 | 137 |
| p58.2.2 | 0.8 | 25 | 3160 | 0.06 | 0.3 | 545 |

The measurement of phosphorylated p58.2 peptide binding to GST-SHP-1.SH2(N) and GST-SHP-1.SH2(C) fusion proteins was at a constant 5 μl/min flow rate. In the representative experiment of which results are reported in the above table I, 130 and 160 RU of respectively p58.2.1 and p58.2.2 peptides were immobilized on microchips. The regeneration was performed using HBS buffer supplemented with 0.03% SDS. Results are expressed as corrected resonance units (CRU) corresponding to the raw RU values subtracted from background RU value due to the injection medium. In this representative experiment, 130 RU of p58.2.1 peptide and 160 RU of p58.2.2 peptide were immobilized on microchips. k_{off} and kₒₙ were calculated from three independent measurements using the BLAevaluation 2.0 software. Kd was calculated from k_{off}/kₒₙ.

However, striking differences were observed between the binding capacities of the isolated SH2 domains. Measurement of kinetic constants revealed that the affinity of the phosphorylated N-terminal KIR ITIM peptide for SHP-1.SH2(C) is 3-3.5 times higher than for SHP-1.SH2(N) (see Table 2). This difference is the direct consequence of a dramatically higher k_{off}, despite an higher kₒₙ, in the interaction of the phosphorylated KIR ITIM peptide with SHP-1.SH2(N) as compared to SHP-1.SH2(C). The N- and C-SH2 domains of SHP-1 exert distinct regulatory roles on SHP-1: whereas the C-H2 domain merely acts as a recruiting unit, the N-SH2 domain not only serves as a docking unit but also as a regulator for SHP-1 PTPase activity.

Therefore, our results showing that KIRs associate with both SHP-1 N- and C-SH2 domains, are in agreement with their reported role as activators of SHP-1 PTPase function. These data also confirm that the N-terminal KIR ITIM bind SHIP-1.SH2(N) and SHP-1.SH2(C) domains more efficiently than the C-terminal KIR ITIM, and support the observation showing that the N-terminal KIR ITIM is sufficient for mediating KIR inhibitory function.

Nevertheless, the binding of both KIR ITIMs to SHP-1 SH2 domains, is reminiscent of the association between SHP-2 SH2 domains and two distinct IRS-1 amino-acid stretches surrounding tyrosine residues 1172 and 1222. The crystallographic analysis of SHP-2 SH2 domain structure revealed that the distance between tyrosine 1172 and tyrosine 1222 is critical for the simultaneous association of both IRS-1 binding sites to SHP-2.SH2(N) and SH-2.SH2 (C), which leads to a dramatic increase in SHP-2 PTPase activity. Since the tyrosine residues present in the N- and C-terminal KIR ITIMS are separated by 30 amino-acids (tyrosine 303 and tyrosine 333 respectively), it is possible that this distance may be sufficient to allow a simultaneous binding of both KIR ITIMs to the N- and C-SH2 domains of SHP-1 and SHP-2 PTPases.

In summary, our data contribute to define the structure of ITIMs, in which the position tyrosine-2 appears to be critical for the binding of SH2-containing PTPases, but not for the binding of SHIP. They also reveal that ITIM-bearing negative coreceptors recruit distinct sets of SH2-containing phosphatases and use divergent strategies in order to mediate their inhibitory function.

### EXAMPLE 3

Inhibition of antigen-induced T cell response and antibody-induced NK cell cytotoxicty by NKG2A: association of NKG2A with SHP-1 and SHP-2 protein-tyrosine phosphatases

ABBREVIATIONS

| | |
|---|---|
| GST: | Glutathione S-transferase. |
| ITAM: | Immunoreceptor tyrosine-based activation motif. |
| ITIM: | Immunoreceptor tyrosine-based inhibition motif. |
| KIR: | Killer-cell inhibitory receptor. |

### SUMMARY

Subsets of T and NK lymphocytes express the CD94-NKG2A heterodimer, a receptor for MHC class I molecules. We show here that engagement of the CD94-NKG2A heterodimer inhibits both antigen-driven TNF release and cytotoxicity on melanoma-specific human T cell clones. Similarly, CD16-mediated NK cell cytotoxicity is extinguished by cross-linking of the CD94-NKG2A heterodimer. Combining in vivo and in vitro analysis, we report that both I/VxYxxL Immunoreceptor Tyrosine-based Inhibition Motifs (ITIMs) present in NKG2A intracytoplasmic domain associate upon tyrosine phosphorylation with the protein tyrosine phosphatases SHP-1 and SHP-2, but not with the polyinositol phosphatase SHIP. Determination of K_{D}, using surface plasmon resonance analysis, indicates that NKG2A phospho-ITIMs directly interact with the SH2 domains of SHP-1 and SHP-2 with a high affinity. Engagement of the CD94-NKG2A heterodimer therefore appears as a protein-tyrosine phosphatase-based strategy that negatively regulates both antigen-induced T cell response and antibody-induced NK cell cytotoxicity. Our results suggest that this inhibitory pathway sets the threshold of T and NK cell activation.

### INTRODUCTION

The control of lymphocyte activation is ensured by a dynamic equilibrium between activatory and inhibitory signals. In particular, antigen-MHC complex and antibody-coated target cells serve as activatory signals for T and NK lymphocytes, and are recognized by the T cell receptor (CD3/TCR) and the CD16 receptor (FcγRIIA) respectively. These oligomeric complexes are coupled to downstream signaling pathways via polypeptides such as CD3γ, CD3δ. CD3ε, CD3ζ and/or FcεRIy for CD3/TCR, as well as CD3ζ and/or FcεRIγ for FcγRIIIA. These polypeptides include in their intracytoplasmic domain one to three immunoreceptor tyrosine-based activation motifs (ITAMs), which are necessary and sufficient for their transduction properties. ITAMs are defined by a consensus YxxL/Ix₆₋₈YxxL/I amino-acid stretch. Reciprocally, inhibitory signals can be provided by engagement of a variety of cell surface receptors, which are characterized by the presence of one or two immunoreceptor tyrosine-based inhibition motifs (ITIMs) in their intracytoplasmic domains. ITIMs are defined by a consensus L/I/VxYccL/V amino-acid stretch. In both T and NK lymphocytes, ITIM-bearing receptors include multigenic families of inhibitory receptors for MHC class I molecules, such as Killer-cell Inhibitory Receptors (KIRs) (human KIRs and the mouse lectin-like Ly-49 molecules) ITIMs present in KIRs and Ly-49 molecules recruit upon tyrosine phosphorylation, the tandem SH2-containing protein tyrosine phosphatase, SHP-1 as well as SHP-2. Similarly to Ly-49 molecules, the human lectin-like NKG2A molecules have been described to serve as inhibitory receptors for MHC class I molecules on NK cells. NKG2A are expressed as heterodimers with another lectin-like molecule, CD94, on both T and NK lymphocytes. Previous study suggested that CD94-NKG2A heterodimer function as inhibitory receptors on CTLs. Here we show that both TCR-induced cytolysis and lymphokine production are down regulated by signaling via the CD94-NKG2A receptor on melanoma-specific T cell clones. We also investigated the mechanisms leading to the inhibitory function exerted by the CD94-NKG2A heterodimers and show that NKG2A express two functional ITIMs that recruit both SHP-1 and SHP-2 protein tyrosine phosphatases via their SH2 domains. Therefore, the CD94-NKG2A heterodimer serves as an ITIM-bearing receptor which control both antigen- and antibody-mediated T and NK cell response respectively.

### MATERIALS AND METHODS

Peptides and Antibodies

ITIM and ITAM peptides were synthesized in phosphorylated (^{\mathring{P}}) and in non-phosphorylated forms, and contain an N-terminal-biotin (Table 3 below).

TABLE 3
List of peptides used in this study

| PEPTIDE | SEQUENCE |
|---|---|
| p58.183.1 (N-terminal | DEQDFQEVTY₃₀₃AQLNH |
| ITIM): | |
| p58.133.2 (C-terminal | RP SQ RPKTPPTDIIVY₃₃₃TELPNAEP |
| ITIM): | |
| FcyII 3 | KTEAENTITY₂₆₂SLK |
| NKG2A N-term (N- | MDNQGVIY₈ SDLNL |
| terminal ITIM): | |
| NKG2A C-term (C- | ILATEQEIT₄₀ AELNL |
| terminal ITIM): | |
| Ly-49A | MSEQEVTY8 SMVRF |
| TYR (tyrosinase | Y₁MDGTMSQV₉ |
| peptide): | |
| EAA (Melan-A/MART-1 | E₂₆AAGIGILTV₃₅ |
| peptide): | |

The p58.183.1 and p58.183.2 peptides were generated from the p58.2 (CD158b) sequence. The FcγRIIB peptide was generated from the murine FcγRIIB1/B2 sequence. Amino-acids are numbered according to the first N-terminal amino-acid of the reported sequences. Melan-A/MART-1 peptide and tyrosinase peptide were purchased from Genosys (Lake Front Circle, USA) and were >70% pure as indicated by analytic HPLC. The generation of 712, an anti-phospho-ITIM antiserum has been previously reported. The anti-SHIP rabbit antiserum was generated using GST-SHIP.SH2 fusion protein as an immunogen. The horseradish peroxidase-conjugated goat anti-rabbit antiserum was purchased from Sigma Chemical Co. The following mouse mAbs have been described elsewhere; anti-CD94 (XA-185, IgGI: HP-3B1, IgG2a), anti-NKG2A (Z199, IgG2b), anti-CD16 (KD1, IgG2a) and anti-CD3e (IKT3, IgG2a). The Z199 mAb recognizes both NKG2A and NKG2B molecules, as these molecules are highly homologous alternative-spliced products of the same gene.

Cells

The following cell lines have been described previously: the mouse mastocytoma cell line P815, the murine B cell lymphoma, IIA.1.6, the IL-2-dependent human NK cell line, NKL and the Rat Basophilic Leukemia line, RBL-2HG. M117-14 and M77-84 CTL clones were derived from melanoma tumor infiltrating lymphocytes (TIL) by limiting dilution culture as described previously. The CTL clone 7-10 was derived from healthy donor PBL stimulated in vitro by the Mclan-A/MART-1 peptide 27-35 and then cloned by limiting dilution as tumor-infiltrating lymphocytes (TILs). Specificity and restriction were investigated using various functional assays including TNF production and cytolytic assays against peptide pulsed target cells and melanoma cells. The three clones are HLA-A*0201 restricted. 7-10 and M77-84 clones recognize the Mclan-A/MART-1₂₆₋₃₅ peptide (EAA peptide) and the M117-14 clone recognizes the Tyrosinase₁₋₉ peptide (TYR peptide).

Fusion Proteins and Surface Plasmon Resonance Analysis

Surface plasmon resonance measurements were performed on a BIAcore apparatus (BIAcore). The GST-SHP1.SH2(NC), GST-SHP2.SH2(NC) and GST-SHIP.SH2 fusion proteins generated from the murine phosphatase cDNAs, were purified from DH5α lysates as previously described. Before their use in BIAcore experiments, fusion proteins were dialyzed in HBS buffer pH 7.4 (10 mM HEPES, 150 mM NaCl, 3.4 mM EDTA). Protein purity was assessed by 12.5% SDS-PAGE, and Coomassie blue staining. The running buffer used in all BIAcore experiments consisted of HBS buffer supplemented with 0.05% surfactant P20. Equilibrium constant determination (k_{off} and kₒₙ) was performed using BIAevaluation 2.0 software. The equilibrium dissociation constants K_{D} were calculated from the k_{off}/kₒₙ ratio.

³⁵S Metabolic Labelling

250×10⁶ IIA.1.6 cells were washed twice by resuspending in methionine and cysteine free RPMI warmed at 37° C. Cells were then resuspended in labeling medium (methionine and cysteine free prewarmed RPMI medium supplemented with 10% FCS. 1% glutamine, 100 V/mi penicillin, 100 μg/ml streptomycin, 5 mM sodium pyruvate, 25 mM HEPES, 50 μM β-mercaptoethanol), and incubated for 45 minutes at 37° C. After centrifugation, cells were resuspended in 150 ml of labeling medium containing 3 mCi Tran³⁵Ser label and 1 mCi³⁵ Cys (ICN), and incubate overnight at 37° C. Cells were washed twice using cold PBS and precleated by three incubations of 1 hour with control peptide (non-phosphorylated CD3ε peptide) immobilized to streptavidin-agarose beads (Sigma) prior their use in the cell lysates adsorption assay.

Assay for Cell Lysates Adsorption to Peptides

RBL-2H3 and ³⁵S-labeled IIA.1.6 cells were lysed in NP-40 lysis buffer (1% NP-40, 10 mM Tris-HCl, 150 mM NaCl, 1 mM EDTA, 1 mM PMSF, 10 mM iodoacetamide, 10 mM NaF, 10 mM Na pyrophosphate, 0.4 mM Na vanadate, 10 μg/ml leupeptin, 10 μg/ml aprotinin). Samples were either used directly (whole cell lysates: WCL), or subjected to affinity purification using peptides bound to beads. Biotinylated peptides (5 μg) were coupled to 50 μl streptavidin-agarose slurry beads for 1 hour at 4° C., prior to bead saturation with D-biotin (1 mg/ml) for 1 hour at 4° C. After 3 washes in lysis buffer, samples were fractionated on 8% SDS-polyacrylamide gel electrophoresis (SDS-PAGE) under reducing condition and transferred to nitrocellulose. Immunoblotting was then carried out with anti-SHP-1 mAb (0.5 μg/ml), anti-SHP-2 mAb (0.5 μg/ml) (Transduction laboratories) or anti-SHIP antiserum, and either horseradish peroxidase-conjugated goat anti-rabbit or horseradish peroxidase-conjugated goat anti-mouse antisera (Sigma) and revealed using the Renaissance chemiluminescence kit (NEN).

Cell Activation and Immunoblotting

Cells were washed 3 times in cold PBS, resuspended at 5×10⁶ cells/ml in PBS and pre-incubated for 15 minutes at 37° C. Cells were then incubated for 15 minutes in the presence or absence of pervanadate (500 μM) prepared as described. Cells were immediately lysed in NP-40 lysis buffer for 15 minutes on ice. After removing insoluble material by centrifugation at 12,000 rpm for 15 minutes, samples were either used directly (WCL:whole cell lysates) or subjected to immunoprecipitation for 45 minutes using indicated mAbs coupled to protein G sepharose beads (Pharmacia). Samples were then combined with reducing sample buffer (New England Biolabs), boiled, prior to fractionation on 8% SDS-PAGE. Immunoblotting was then carried out using anti-SHP-1 mAb, anti-SHP-2 mAb, anti-SHIP antiserum and 712 anti-phospho-ITIM antiserum, in parallel or successively. Nitrocellulose filters (Schleicher & Schull) were then incubated either with horseradish peroxidase-conjugated goat anti-rabbit antiserum or horseradish peroxidase-conjugated goat anti-mouse antiserum (Sigma), and the chemilumincscence was detected using the Renaissance chemuiluminescence kit.

Cytotoxicity Assays

The cytolytic activity of NKL cells and CTL clones was assessed against the B815 mastocytoma mouse cell line in the presence or absence of indicated mAb. Briefly, 5×10^{3 51}Cr-labeled target cells were added to serial dilutions of NKL cells in the presence of indicated mAb at the initiation of a standard 4 hour ⁵¹Cr-release assay. In parallel, 1×10^{3 51}Cr-labeled target cells were added to 1×10⁴ CTL clones in the presence of serial dilutions of purified anti-CD3ε mAb at the initiation of a standard 4 hour ⁵¹Cr-release assay. Except for the anti-CD3ε mAb, mAbs were used as crude hybridoma supernatant (50 μl) for a 150 μl final volume.

Auto-presentation Assay

5×10⁴ CTLs were incubated with indicated concentrations of peptides in 100 μl final volume. After two hours, supernatants were harvested to test the TNF secretion and lysis was estimated by flow cytometry on the basis of size/granularity patterns as previously described. TNF determinations were performed by a biologic assay using cytotoxicity on the sensitive WEHI 164 clone 13 cells, as compared to a standard curve established using rTNF-β (Genzyme).

### RESULTS

Expression and Inhibitory Function of the CD94-NKG2A Heterodimer on NK and T Cells

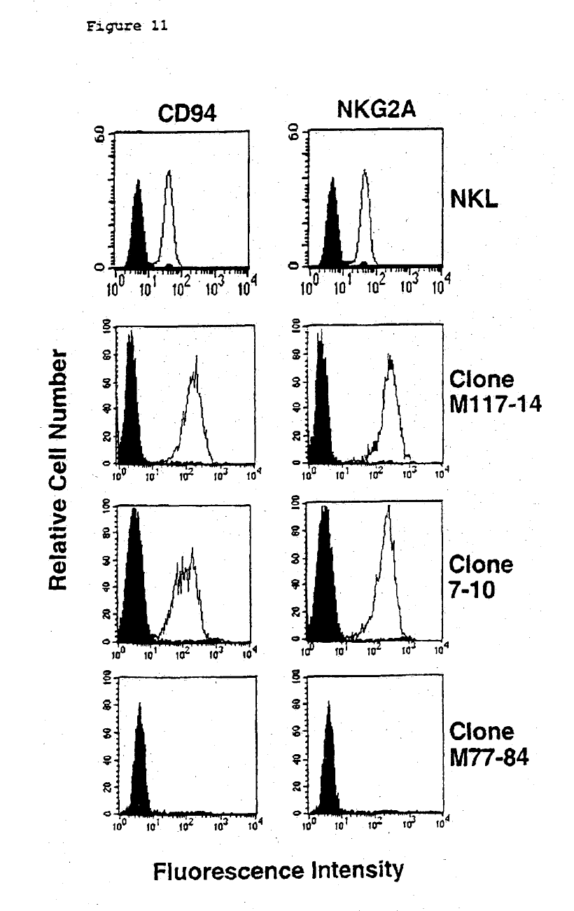

It has been recently reported that a melanoma-specific TCRαβ⁺ CTL clone expresses an NK inhibitory receptor p58.2 KIR (CD158b), which inhibits its cytolytic function. We systematically analyzed a panel of 13 melanoma specific TCTαβ⁺ CTL clones for the expression of Ig superfamily KIR p58, p70 and p140. In addition, we documented the expression of the lectin-like molecules NKG2A and CD94, as CD94 is expressed by some T lymphocyte subsets and was recently shown to be included in a heterodimer with NKG2A on NK cells. Two out of thirteen CD8⁺ TIL clones specific for autologous tumor cells express the CD94-NKG2A heterodimers. FIG. 11 shows the expression of NKG2A and CD94 on one of these TIL clones, M117-14, and on clone 7-10 (derived from PBL), as well as the absence of both molecules on another TIL clone M77-84.

In FIG. 11, NKL cells and the TCRαβ⁺CD8⁺ CTL clones (M117-14, 7-10, M77-84) were analyzed by indirect immunofluorescence and flow cytometry using a FACScan apparatus as described. (32). The empty histograms show staining with anti-CD94 mAb (XA-185 or HP-3B1) or anti-NKG2A mAb (Z199), while the filled histograms represent negative control staining (irrelevant mouse IgG).

The CD94-NKG2A⁺ CTL clones were also characterized by their weak cytolytic activity against autologous target tumor cell lines as compared to allogeneic melanoma cell lines; yet, both autologous and allogenic melanoma-cells expressed comparable levels of the restricting HLA-A*0201 molecule at their surface, and present similar amounts of antigen, as assessed by semi-quantitative RT-PCR and FACS analysis respectively. In parallel, the expression of the CD94-NKG2A heterodimer was confirmed on the surface of the human IL-2-dependent NK cell line, NKL (see FIG. 11). Immunoprecipitation analysis using anti-NKG2A and CD94 mAb confirmed that all detectable NKG2A and CD94 molecules are associated in a CD94-NKG2A heterodimer at the surface of M117-14, 7-10 and NKL cells. Interestingly, the NKG2A-CD94⁺ M117-14 and 7-10 CTL clones as well as NKL cells do not express any p58, p70 or p140 KIRs recognized by EB6, GL183, Z27 or NKB1, and DEC66 mAbs respectively. The function of the CD94-NKG2A heterodimers on NK cells and CTL clones was then investigated by several approaches. In a first set of experiments, anti-CD3 and anti-CD16 mAbs redirected killing assays against the FeγR⁺ murine cell line P815 were performed on CTL and NKL cells respectively, in the absence or presence of anti-CD94 or anti-NKG2A mAbs.

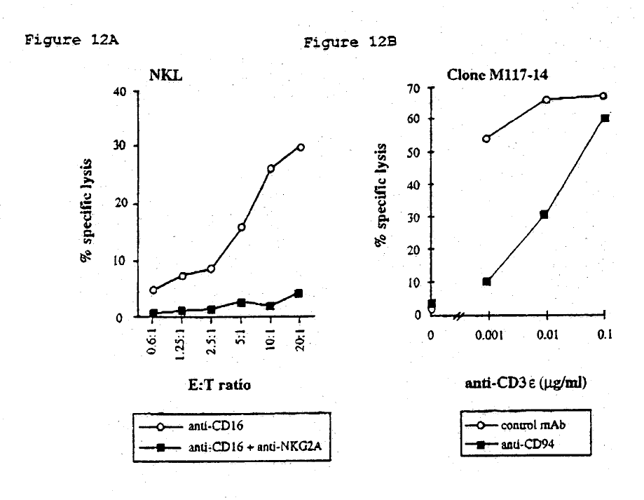

Results are illustrated by FIG. 12 showing that the CD94-NKG2A engagement inhibits cytotoxicity on NKL cells and melanoma specific T-cell clones. In FIG. 12, NKL cells and T cell clones were used as effector cells in a 4 hour mAb-redirected killing assay against P815 cells. (12A) For NKL cells, this assay was performed in the presence of anti-CD16 mAb (open circles) or anti-CD16+anti-NKG2A mAbs (filled squares). No cytotoxicity was detected when NKL and P815 cells were incubated in the absence of mAb, as well as in the presence of anti-NKG2A or anti-CD94 mAbs alone. (12B) P815 cell lysis induced by the melanoma-specific CTL clone M117-14 was generated by anti-CD3ε mAb. This assay was performed in the presence of anti-CD94 mAb (HP-3Bl, filled squares) or anti-CD19 mAb (control mAb, IgG2a, open circles) at an effector to target ratio (E:T) of 10:1.

An efficient inhibition of the CD16-dependent NKL cell cyolytic activity was achieved following cross-linking of NKG2A (FIG. 12A), and similar data were obtained upon cross-linking of CD94, Anti-CD3 mAb-induced lysis of P815 cells by CD94-NKG2A⁺M117-14 cells was also inhibited by cross-linking of CD94 (FIG. 12B) or NKG2A, whereas an irrelevant IgG had no effect. Similar data were obtained on CD94-NKG2A⁺7-10 cells, whereas no effect of either anti-NKG2A or anti-CD94 mAbs was detected when anti-CD3 mAb redirected killing assays were performed on the CD94-NKG2A⁻M77-84 CTL clone. Interestingly, engagement of the CD94-NKG2A heterodimer fails to inhibit T cell redirected killing of P815 cells induced by supra-optimal concentrations (≧0.1 μg/ml) of anti-CD3 mAb (FIG. 12B), consistent with the inhibitory function described for KIRs.

Inhibition induced by the CD94-NKG2A heterodimer on antigen-specific T cell activation

We then analyzed the involvement of the CD94-NKG2A heterodimer in the antigen-specific response of M117-14 and 7-10 melanoma-specific CTL clones. To this end, we used an auto-presentation assay. In the presence of cognate peptides, i.e. Tyrosinase₁₋₉ peptide (TYR peptide) for clone M117-14 and Melan-A/MART-1₂₆₋₃₅ peptide (EAA) for the other two clones, CTLs are lysed in a fratricidal pathway and secrete TNF. In these experiments, anti-CD94 and anti-NKG2A mAbs were used in the absence of cross-linking to mask the CD94-NKG2A heterodimer.

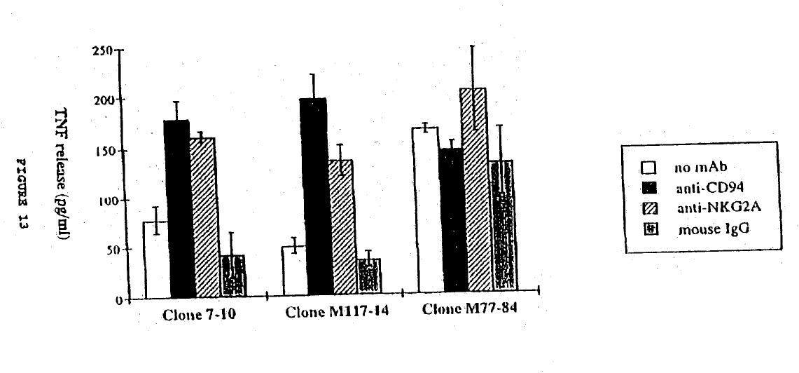

Results are illustrated by FIG. 13 showing that CD94-NKG2A inhibits the antigen-specific TNF production by CTL clones. In FIG. 13, the melanoma specific CTL clones M117-14 and 7-10 were stimulated in an auto-presentation assay with their respective cognate peptides (i.e.:EAA for 7-10 and M77-84, and TYR for M117-14) at a concentration of 10 μM, in the presence or indicated mAbs. TNF release was assessed in the supernatant by a biological assay using the WEHI 164, a TNF sensitive cell line. Data from one representative experiment out of five, are expressed as mean TNF (pg/ml)±SD of triplicate samples.

As shown in FIG. 13, M117-14 and 7-10 released TNF upon cognate antigenic stimulation, i.e. following exposure to TYR and EAA peptides respectively. The antigen-specific stimulation of M117-14 and 7-10 in the presence of anti-CD94 or anti-NKG2A mAbs resulted in a significant increase in TNF production (FIG. 13). As controls, the addition of an irrelevant mouse IgG did not modify the TNF release induced upon antigen-specific stimulation on clones M117-14 and 7-10; in addition, anti-CD94 or anti-NKG2A mAbs did not alter the production of TNF induced upon antigen stimulation (EAA peptide) of the CD94-NKG2A⁻M77-84 CTL clone. We further investigated whether the CD94-NKG2A heterodimer was also involved in the control of antigen-induced CTL cytotoxicity.

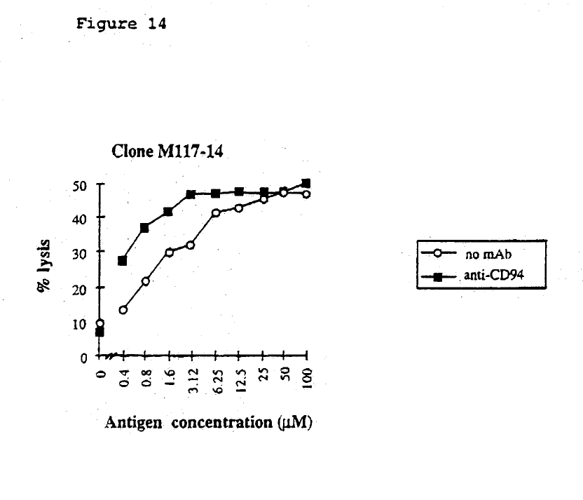

Results are illustrated by FIG. 14 showing the negative regulation of antigen-induced CTL clone cytotoxicity by CD94-NKG2A. In FIG. 14, cells from the melanoma specific CTL clone M117-14 were stimulated in an auto-presentation assay with the indicated concentrations of cognate peptide (TYR). Cytotoxicity was measured, in the presence or absence of anti-CD94 mAb (HB-3B1). Cell lysis was estimated by flow cytometry on the basis of size/granularity patterns after auto-presentation in a short term assay (2 hours). Data shown are representative from 5 independent experiments.

As shown in FIG. 14, M117-14 cells are lysed in a dose-dependent manner, in the presence of increasing concentrations of TYR cognate peptide. Similarly to the effect observed on TNF release, addition of anti-CD94 (FIG. 14) or anti-NKG2A mAbs resulted in a significant increase in CTL cells auto-toxicity. A comparable increase in CTL lytic activity was obtained with both mAbs using clone 7-10. Therefore, in the absence of cross-linking, the use of anti-NKG2A and anti-CD94 mAbs blocks the interaction between the CD94-NKG2A heterodimer and its MHC class I ligand, and further reveals an endogenous negative regulation exerted by the CD94-NKG2A heterodimer on CTL activation induced by melanoma antigens. However, no effect of anti-CD94 mAb was detected at supra-optimal concentrations of cognate antigenic peptides, i.e. ≧25 μM for M117-14 (FIG. 14), consistent with the failure of CD94-NKG2A to modulate T cell activation induced by supra-optimal concentrations of anti-CD3 mAb (FIG. 12B).

NKG2A is an ITIM-bearing molecule

We then analyzed the mechanisms used by the CD94-NKG2A heterodimer to inhibit T and NK cell activation. The intracytoplasmic domain of CD94 only includes 7 amino-acids, and is devoid of any characteristic motif coupled to transduction pathways. By contrast NKG2A is characterized by the presence in its intracytoplasmic domain of two I/VxYxxL motifs which are consensus to ITIMs (ViYsdL and ItYael, for the N- and the C-terminal motifs respectively). ITIMs are functional upon phosphorylation of the tyrosine residue. In an attempt to characterize the function of these putative ITIMs, cell lysates were incubated with phosphorylated and nonphosphorylated synthetic peptides corresponding to the N-terminal and the C-terminal I/VxYxxL stretches present in NKG2A intracytoplasmic domain. Cell lysates absorbed to peptides were then assayed by immunoblotting for the presence of the phosphatases known to interact with phosphorylated ITIMs, i.e. the protein tyrosine phosphatases SHP-1 and SHP-2, as well as the polyinositol phosphate phosphatase, SHIP. In parallel, lysates adsorption were performed using control peptides corresponding to ITIMs present in Ig-like ITIM bearing receptors such as human KIRs (p58.2/CD158b, a receptor for HLA-Cw3) and mouse FcγRIIB, as well as in mouse lectin-like ITIM-bearing receptor, such as Ly-49A.

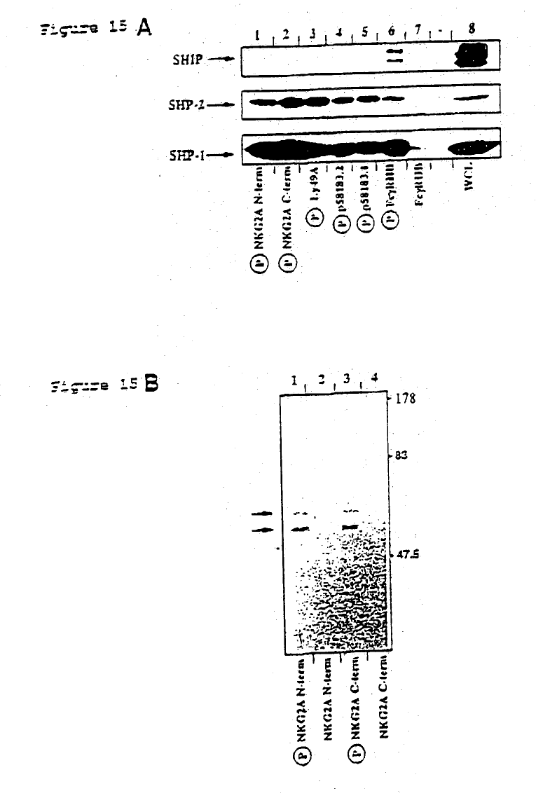

Results are illustrated by FIG. 15 showing the in vitro interaction between NKG2A ITIMs and SHP-1, SHP-2 and SHIP phosphatases. In FIG. 15, RBL-2H3 cell lysates (15A) or ³⁵S-labeled ILA.1.6 cell lysates (15B) were absorbed with indicated biotinylated peptides coupled to streptavidin-beads. Affinity-bound proteins (30×10⁶ cells/sample) or whole cell lysates (WCL, 5×10⁶ cells/sample) were resolved on 8% SDS-PAGE under reducing conditions prior to autoradiography (15B), or immunoblotting using anti-SHIP antiserum, anti-SHP-2 mAb and anti-SHP-1 mAbs (15A).

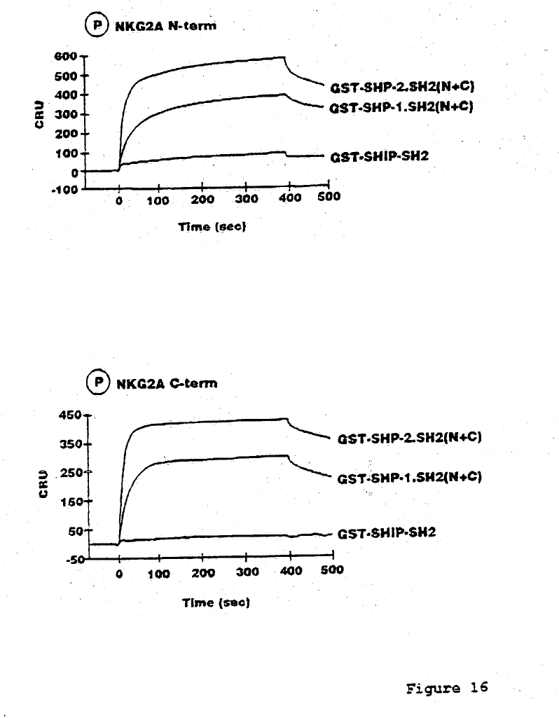

As shown in FIG. 15A (lanes 1 and 2), both N- and C-terminal I/VxYxxL stretches present in NKG2A associate with SHP-1 and SHP-2 in vitro, but no binding to SHIP was detected. Similar patterns of association were obtained using the phosphorylated ITIM peptides of Ly-19A and p58.2 KIR (FIG. 15A, lanes 3 to 5). In contrast, FcγRIIB phosphorylated ITIM peptides also associate with SHIP (FIG. 15A, lane 6). No association of phosphatases with nonphosphorylated peptides corresponding to FcγRIIB (FIG. 15A, lane 7) and NKG2A N- and C-terminal I/VxYxxL stretches was detected. When nonphosphorylated and phosphorylated peptides were used to adsorb lysates prepared from ³⁵S methionine/cysteine-labeled cells, only two bands around 64 and 68 kDa selectively associate with both phosphorylated NKG2A N- and C-terminal I/VxYxxL stretches, as compared to nonphosphorylated ones (FIG. 15B). The 64 and 68 kDa proteins which bind to phosphorylated NKG2A peptides correspond to SHP-1 and SHP-2 apparent molecular weight respectively, supporting the immunoblotting results. Therefore, our results define the two NKG2A I/VxYxxL stretches as ITIMs which appear to function similarly to other ITIMs present in inhibitory receptors for MHC class I molecules, i.e. KIRs and Ly-49 molecules. Using recombinant soluble tandem SH2 domains of SHP-1 and SHP-2, we confirmed by surface plasmon resonance that both NKG2A ITIM phosphopeptides directly associate with the SH2 domains of SHP-1 and SHP-2, but not with the SH2 domain of SHIP (FIG. 16).

Results are illustrated by FIG. 16 showing the BIAcore analysis of NKG2A ITIM interaction with the SH2 domains of SHP-1, SHP-2 and SHIP phosphatases. In FIG. 16, the binding of 100 nM soluble recombinant GST-SHP-1.SH2(N+C), GST-SHP-2.SH2(N+C) or GST-SHIP.SH2 to immobilized NKG2A/B phosphorylated ITIM peptides (25 RU) was monitored by real-time analysis using surface plasmon resonance. Results are expressed as corrected resonance units (CRU) corresponding to the raw RU values after subtraction of background RU value due to the injection medium.

We further assessed that the interactions between NKG2A phosphorylated ITIM peptides and the phosphatase SH2 domains follow a first order reaction (Legends to the below Table 4).

TABLE 4
Association and dissociation constants for the interaction of
SHP-1..SH2(N + C) and SHP-2.SH2(N + C)
domains with phosphorylated NKG2A ITIM peptides.

| Phospho- | GST-SHP-1.SH2(N + C) | | | GST-SHP-2.SH2(N + C) | | |
|---|---|---|---|---|---|---|
| rylated | kₒₙ | k_{off} | K_{d} | kₒₙ | k_{off} | K_{d} |
| peptides | (10⁻⁶M⁻¹s⁻¹) | (10³s⁻¹) | (nM) | (10⁻⁶M⁻¹s⁻¹) | (10³s⁻¹) | (nM) |
| NKG2-A | 0.39 | 1.89 | 4.80 | 1.03 | 1.42 | 1.38 |
| N-terminal | | | | | | |
| ITIM | | | | | | |
| NKG2-A | 0.41 | 1.17 | 2.83 | 0.84 | 1.32 | 1.57 |
| C-terminal | | | | | | |
| ITIM | | | | | | |

The measurement of phosphorylated peptide binding to GST-SHP-1.SH2(N+C) and GST-SHP-2.SH2(N+C) fusion proteins was performed at a constant 5 μl/min flow rate. In this representative experiment, 25 RU of phosphorylated NKG2A N-term and NKG2A C-term peptides were immobilized on streptavidin microchips. The regeneration was performed using HBS buffer supplemented with 0.03% SDS. Results are expressed as corrected resonance units (CRU) corresponding to the raw RU values after subtraction of background RU value due to the injection medium, k_{off} and kₒₙ were calculated from three independent measurements using the BIAevaluation 2.0 software. In addition, kₒₙ was calculated as the slope of the curve k_{g}=kₒₙ×c−k_{off}, where k_{off} is the off-rate constant and c is the concentration of the soluble recombinant GST fusion proteins. By plotting k_{g} as a function of c, a linear regression fit was obtained (r²>0.98) for the binding of NKG2A ITIM N- and C-term to SHP-1 and SHP-2 tandem SH2 domains. This linear representation allows to check on the validity of the single step interaction and to confirm the determination of the kₒₙ constant using non-linear analysis (BIAevaluation software).

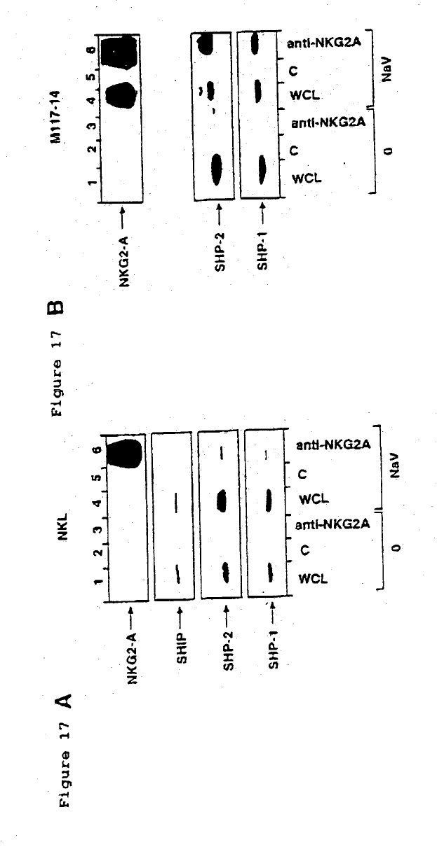

As a consequence, the K_{D} which characterize these associations ere determined, and were shown to vary between 1 and 5 nM (Table 4). These data show that high affinity interactions exist between SHP-1 or SHP-2 and both phosphorylated NKG2A ITIMs. We then analyzed the association of NKG2A with SHP-1 and SHP-2 in vivo. We used the 712 anti-phosphol ITIM antiserum to first probe the tyrosine phosphorylation of NKG2A. The 712 antiserum is an anti-phosphotyrosine antiserum that selectively reacts with phosphorylated ITIMs. NKL cells and M117-14 cells were stimulated or not by pervanadate, which induces a general increase in the catalytic activity of protein tyrosine kinases. Anti-NKG2A immunoprecipitations prepared from pervanadare-stimulated cells include a phosphoprotein, which migrates at ˜43 kDa under reducing conditions (FIG. 17).

Results are illustrated by FIG. 17 showing the in vivo recruitment of SHP-1 and SHP-2 by phosphorylated NKG2A. In FIG. 17, NKL cells (17A) and M117-14 cells (17B) were stimulated or not using pervanadate (NaV, 500 μM). Cell lysates were separated by 8% SDS-PAGE under reducing conditions either directly (WCL; 2×10⁶/sample for KNL and 2.5×10⁶/sample for M117-14) or after immunoprecipitation using indicated mAb (100×10⁶/sample for NKL and 120×10⁶/sample for M117-14), transferred to nitrocellulose and immunoblotted using anti-SHP-1 mAb, anti-SHP-2 mAb as well as anti-SHIP and 712 anti-phospho-ITIM antisera. Z199 mAb was used for the anti-NKG2A immunoprecipitations whereas a mouse anti-Vβ8.2 mAb (F23.2, IgGl) was used as a negative control mAb for immunoprecipitations (C).

Since the 43 kDa phosphoprotein is recognized by the 712 antiserum, and both tyrosine residues present in NKG2A intracytoplasmic domain are included in NKG2A ITIMs, these results indicate that NKG2A is tyrosine-phosphorylated on ITIMs upon pervanadate treatment in both T and NK lymphocytes. Moreover, anti-NKG2A immunoprecipitates prepared from pervanadate-stimulated NKL cells and M117-14 cells include SHP-1 and SHP-2 but SHIP (FIG. 17), confirming the recruitment of SHP-1 and SHP-2 protein tyrosine phosphatases by phosphorylated NKG2A ITIMs in vivo. Similarly, the recruitment of SHP-1 and SHP-2 was observed when mAb-induced co-aggregation between CD16 and CD94-NKG2A, or CD3/TCR and CD94-NKG2A, was performed on NKL and M117-14 cells respectively.

Discussion

The negative regulation of lymphocyte activation is central to the homeostasis of the immune response and is also of primary interest to the rationale manipulation of the immune system. We show here that the lectin-like molecule NKG2A is a potent negative regulator of T and NK lymphocyte activation programs (FIG. 12, 13 and 14), raising several points relative to the function as well as the mechanisms of action of the CD94-NKG2A heterodimer. First, we show here the expression of the inhibitory receptor CD94-NKG2A by two TCRαβ⁺CD8⁺ CTL clones specific for melanoma antigens (FIG. 11). It is, to our knowledge, the first report on TCRαβ⁺ T cell clones of known specificity that express this receptor. Previous data stated that CD94 expression is highly restricted to NK and T cell subsets, mostly TCRγδ⁺, which display non MHC-restricted cytotoxicity. The CD94-NKG2A⁺ T cell clones described here exhibited a classical MHC-restricted lysis and lacked NK-like activity, as they were CD16⁻ and did not kill K562 cells. Recently a melanoma-specific CTL clone was shown to express the p58.2 KIR which recognizes HLA-Cw3 and related haplotypes such as HLA-Cw7. This clone recognizes the PRAME antigen on autologous melanoma, but only HLA-Cw7 loss variants of these cells were killed by this CTL, HLA-Cw7 thus appeared as an endogenous ligand for p58.2 KIR on these cells, and triggering of p58.2 by this ligand inhibits tumor cell lysis. Among the 13 melanoma specific CTL clones that we have tested none of them expressed the p58.2 molecule, nor any other p58, p70 or p140 KIRs described so far. However, two of them expressed the CD94-NKG2A receptor at high density. Triggering of this receptor inhibited antigen- and anti-CD3 mAb-induced activation of these clones, lowering both lytic and cytokine responses. Therefore, at least two classes of NK-like inhibitory receptors (NKRs), Ig-like and lectin-like, may be expressed by melanoma-specific CTLs, and the expression of the lectin-like CD94-NKG2A receptor appears as a novel example of an inhibitory strategy which governs melanoma-specific CTL activation. It has been reported that the CD94-NKG2A heterodimer serves as a receptor for a broad range of HLA-class I molecules. The data reported here from antigen auto-presentation experiments indicate that TCRαβ⁺T lymphocytes express both the CD94NKG2A receptor and its MHC class I ligand. Expression of NKRs by melanoma-specific CTLs might be related to the conditions of T cell stimulation inside these tumors. Supporting this hypothesis, IL-15 was shown to favor the expression of the CD94-NKG2A by thymocyte precursor derived NK cells and we have recently detected IL-15 mRNAs in most melanoma lines and melanoma tumors by RT-PCR. It is thus possible that this cytokine is involved in the induction of NKRs by melanoma TILs. Alternatively, the nature of melanoma-specific antigens might also be involved in the expression of NKRs by T cells. In this regard and despite the existence of melanoma-specific antigens, it is noteworthy that the antigens recognized by the p58.2⁺ and by the CD94-NKG2A⁺ melanoma-specific CTL clones are autologous antigens. Indeed, PRAME, Melan-A/MART-1 and the tyrosinase antigens are expressed in melanoma cells, but also in normal tissues. It has been observed that only small fractions of healthy donor PBL, mostly monoclonal or oligoclonal CD8⁺ T cell expansions express NKRs, and that CD8⁺ T cell expansions are more frequent in autoimmune disease. It is thus possible that CD3⁺NKR⁺ lymphocytes could be biased towards recognition of self. Further investigations are needed for a better understanding of the mechanism leading to NKR expression on T cells and to the functional consequences of their inhibitory regulation of T cell function. Nevertheless, if a significant proportion of anti-tumor CTLs express NKRs, the inhibitory properties may contribute to the inefficient control of tumor growth by tumor-specific CTLs, as long as tumor cells express the ligand. This also suggests that similarly to NK cells some tumor specific CTLs could lyse only tumor cell variants having lost the CD94-NKG2A ligand, i.e. MHC class I molecules. Such clones could represent as recently suggested, a new category of anti-tumor CTLs situated between NKR⁻ CTLs and KN cells. Second, regarding to the mechanisms involved in the inhibitory function of the NKG2A molecule, our data demonstrate that NKG2A carries two functional ITIMs which directly recruit in vivo, with a high affinity, the protein tyrosine phosphatases, SHP-1 and SHP-2 (FIG. 15, 16, 17, Table 4). Consistent with our data, ITIM-bearing receptors, such as the CD3TCR and the CD16 complexes. As for other ITIM-bearing molecules, NKG2A is phosphorylated on the tyrosine residue present in ITIMs, and associates with the SH2 domains of the phosphatases. ITIM-bearing molecules can be divided into two groups of molecules depending on the nature of the phosphatase that they recruit, i.e.: protein tyrosine phosphatases SHP-1 or SHP-2, or the polymositrol phosphatase, SHIP (see the above examples). Only a sub-group of low affinity receptors for IgG expressing only one ITIM, the FcγRIIB molecules, have been reported to associate with SHIP in vivo. Other ITIM-bearing molecules associate with SHP-1, and mediate their inhibitory function via the increased activity of the phosphatase. In particular, KIRs and CD22 express two or three ITIMs respectively, and it is likely that two phosphorylated ITIMs expressed on the same molecule, simultaneously interact with both SH2 domains of SHP-1. This hypothesis is supported by the cyrstallographic analysis of SHP-2 tandem SH2 domains, as SHP-2 is related to SHP-1. In addition, SHP-2 catalytic activity is increased as a consequence of the simultaneous binding of phosphotyrosine-containing amino-acid stretches to its N- and C-terminal SH2 domains. NKG2A molecules carry two ITIMs separated by 31 amino-acids (tyrosine 8 and tyrosine 40) similarly to KIRs N- and C-terminal ITIMs which are distant of 30 amino-acids. Therefore, the high affinity of each NKG2A phospholTIM interaction with the isolated SH2 domains (1 to 5 nM, Table 4) could even be enhanced by the simultaneous binding of both NKG2A ITIMs to the tandem SH2 domains of SHP-1. SHP-1 has been shown to dephosphorylate phosphotytrosine proteins involved in CD3/TCR- and in CD16-coupled signaling pathways, i.e. pp36-38, CD3ζ, as well as the p725yk and ZAP-70 tandem SH2 protein tyrosine kinases. The binding of NKG2A to SHP-1 is thus likely to result in an increase in SHP-1 phosphatase activity, as for KIRs and CD22. Although SHP-2 is involved in the positive regulation of a number of pathways, the recruitment of SHP-2 by NKG2A might similarly contribute to the dephosphorylation of a set of phosphoproteins belonging to ITAM-dependent cascades. This hypothesis is supported by the association between CTLA-4 and SHP-2, which appears to be involved in the inhibitory function mediated by CTLA-4. Our data thus strongly suggest that NKG2A utilizes a protein tyrosine phosphatase-based mechanism of inhibition, which is common to other ITIM-bearing receptors except for FcγRIIB. Third, the inhibition of T cell cytotoxicity via the CD94-NKG2A heterodimer is only partial and appears to be overcome when T cell are stimulated by supra-optimal concentrations of either anti-CD3ε mAb or cognate peptides (FIG. 12B and 14). A similar failure of ITIM-bearing receptors to inhibit a supra-optimal cell activation has been reported for other ITIM-bearing molecules such as KIRs and FcγRIIB. These observations are consistent with the requirement of a co-aggregation between ITAM- and ITIM-bearing receptors for the inhibitory function mediated by ITIM-bearing receptors. This general property of ITIM-bearing receptors ensures their selectivity of inhibition, which only occurs for the activatory receptors that are co-aggregated with the inhibitory ones. As a result, activatory receptors which are not co-aggregated with ITIM-bearing receptors are not inhibited. Those stimulation conditions may be mimicked when supra-optimal concentrations of anti-CD3ζ mAb or cognate peptides are used to stimulate M117-14 CTL cells. The low density of CD16 receptors expressed at the surface of NKL cells, as compared to the high level of expression of the TCR on M117-14 cells, likely accounts for the absence of failure of the CD94-NKG2A heterodimer to inhibit anti-CD16-induced NKL cell cytotoxicity. In any event, these results indicate that engagement of the CD94-NKG2A receptor on T cells markedly down-regulates the activatory signals delivered via the TCR by increasing its threshold sensitivity to the cognate antigen concentration.

### EXAMPLE 4

Transgenic mice expressing a human KIR

Transgenic mice were generated using the cDNA encoding for p58.2 (cl. 6), inserted in the HindIII version of the pHSE3′ transgenic vector under the control of the H-2K^{b} promoter. Splenocytes and peripheral blood lymphocytes isolated from p58.2 transgenic animals were analyzed by immunofluorescence and flow cytometry.

The data reveal that the human p58.2 molecule is expressed at the cell surface of both mouse T and NK cells. The p58.2 Ig-like KIR recognizes the HLA-Cw3. Therefore, the cell surface expression of HLA-Cw3 confers the protection of target cells against NK-cell mediated natural cytotoxicity. Using the murine mastocytoma cells P815 transfected (P815-Cw3) or not with the HLA-Cw3 cDNA, it has been observed that NK cells isolated from p58.2 transgenic mice can induce the lysis of parental P815 cells but are inefficient in inducing the lysis of P815-Cw3 cells.

These data show for the first time the functional reconstitution of a human Ig-like KIR in the mouse model.

### Material and Methods

Generation of CD158b Transgenic Mice. The CD158b cDNA (p58.2 cl. 6.11) was subcloned in the MHC class I promoter/immunoglobulin enhancer expression cassette pHSE3′-HinDIII and injected into fertilized C57HL/6 (B6) (H-2^{b/b})×CBA/J (H-2^{k/k}) F₂ eggs. Transgenic founder mice and their transgenic progenies were identified by PCR with primers specific for CD158b cDNA and by immunofluorescence analysis of peripheral blood lymphocytes (PEL) using biotinylated GL183 mAb (anti-CD158b) followed by phycoerythrin-conjugated streptavicin. Transgenic lines were established and maintained by crossing of founders with B6 mice. C57BL/6-HLA-CW3 (H-2^{b/b}) transgenic mice were obtained through standard procedure (Dill et al. 1988, Proc. Natl. Acad. Sci. USA 85, 5664-5668). All the mice used in this study were between 6 and 24 weeks old and were maintained at the Animal Facility of the Centre d'Immunologie de Marseille-Luminy.

Immunofluorescence Analysis. Spleen cells and PBL were stained as previously described and analyzed on a FACSan apparatus (Becton Dickinson). The mAbs used in these experiments have been previously described fluorescein isothiocyanate (FITC)-conjugated anti-CD3ζ (Pharmingen), F4/326 (anti-HLA-C), biotinylated GL183 (anti-CD158b), as well as biotinylated anti-human CD2 and FITC-conjugated anti-human CD3, both used as negative controls (Immunotech, Marseille, France). 11.4.1 (anti-H-K2^{k}) and 20.8.4 (anti-H-2K^{b}) mAbs were used for the determination of the H-2 haplotype. Indirect immunofluorescence staining was carried out with FITC- or phycoerythrin-conjugated secondary antibodies of the appropriate species and isotype specificity, purchased from Southern Biotechnology Associates; tricolor (TC)-conjugated streptavidin was purchased from Caltag (South San Francisco, Calif.) and phycoerythrin-conjugated streptavidin from Immunotech.

Cytolytic Assay. To increase the number of splenic NK cells, mice were injected i.p. with 200 μg of poly(I:C) (Pharmacia) 24 hr prior to sacrifice. Spleens were then harvested and single cell suspensions were prepared in RPMI 1640 medium containing 10% fetal calf serum. Erythrocytes were depleted by osmotic lysis and macrophages were removed by 1 hr adherence step on 6-well plates at a concentration of 5×106 cells/ml. These freshly isolated nonadherent splenocytes were used as effector cells in a 4-hr ³¹Cr Release assay. The NK sensitive YAC-1 cell line, the murine mastocytoms cells line P815 [parental (221) as well as transfected with the HLA-Cw3 allele), and the human cell line LCL 721.221 (parental (221) as well as transfected with the HLA-Cw3 (221-Cw3) or HLA-Cw4 (221-Cw4) alleles], were used as target cells. In these assays, 5×10^{3 51}Cr-labeled target cells were added to effector cells at various at various effector: target ratios in V-bottom 96-well plates (final volume 200 μl). After 4 hr at 37° C., 100 μl of supernatant was collected from each well and counted in a γ-counter for the determination of ⁵¹Cr release and percentage specific lysis.

Bone Marrow Grafts. Recipient HLA-Cw3 (H-2^{b/b}) transgenic, HLA-Cw3 (H-2^{k/b}) transgenic, and CD158b X HLA-Cw3 (H-2^{k/b}) transgenic mice were irradiated (950 rads from a ¹²⁷Cs source) and inoculated intravenously with 5×10⁶ T-depleted bone marrow cells from B6 HLA-Cw3 (H-2^{b/b}) transgenic mice. Five days later, recipient mice were injected i.p. with 3 μC. 5-[¹²⁵I] iodo-2′-deoxyuridine (¹²⁵IdUdr, Amersham), Animals were killed 24 hr later and incorporated radioactivity in the spleen was measured in a γ counter.

### Results

Reconstitution of in vitro KIR inhibitory function in NK and T lymphocytes expressing the CD158b transgene.

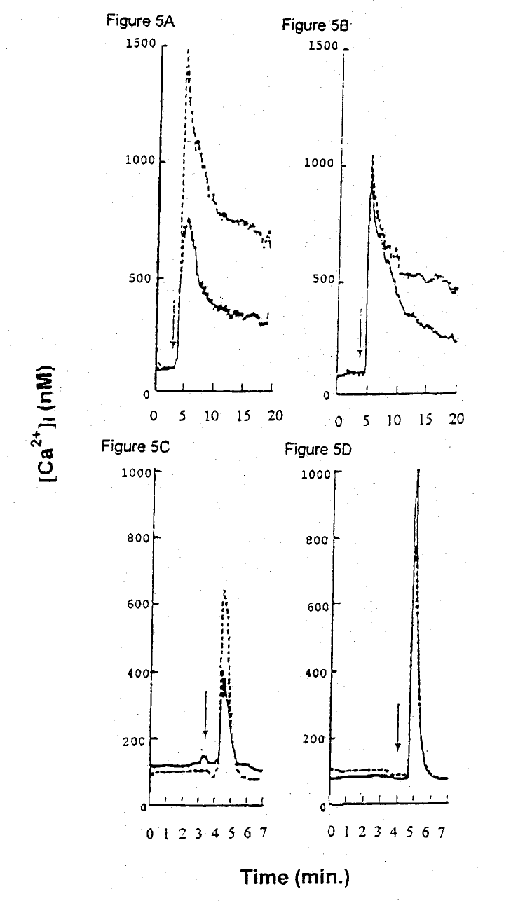

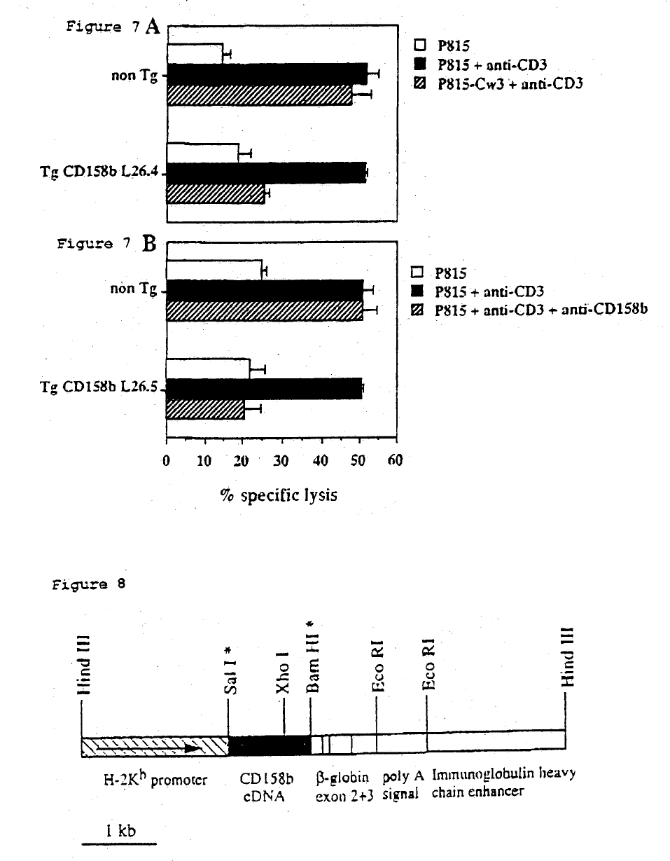

Four foundor mice carrying the CD158b transgene (Tg CD158b) were generated using a MHC class I promoter/immunoglobulin ehancer expression cassette (FIG. 8.) FIG. 8 shows a schematic representation of the CD158b transgenic vector, (the restriction sites marked with an asterisk were destroyed during plasmid construction). Analyses were performed on three independent transgenic lines (L26, L47 and L61) established following stable transmission of the CD158b transgene. In particular, the CD158b transgene was expressed on 85±8% (mean±SEM, n=8) of PBL isolated from the Tg CD158b L61 mice, as determined by flow cytometry. The vast majority of T cells (95±4% of CD3ζ+cells, n=6) and NK cells (78+4% of CD3ζ⁻, sIg⁻, cells, n=3) expressed the CD158b transgene as shown for a representative Tg CD158b L61 mouse in FIG. 6.

FIG. 6 illustrates the cell surface expression of the CD158b transgene. PBL isolated from mice representative of the indicated mouse lines were examined by flow cytometry for the cell surface expression of CD158b, CD3ζ, surface immunoglobulin (sIg), and HLA-Cw3; non transgenic, non-Tg; HLA-Cw3 transgenic, Tg HLA-Cw3; CD158b transgenic, Tg CD158b (L61); HLA-Cw3 and CD158b transgenic, Tg CD158b X HLA-Cw3. Colis were stained with FITC-goat anti-mouse IgC; after saturation of free binding sites with mouse Ig, FITC anti-CD3ζ and biotinylated GL183 (anti-CD158b) mabs were added. Biotinylated GL183 was revealed using TC streptavidin. For HLA-Cw3 expression cells were incubated with F4/326 mAb (anti-HLA-C) followed by a FITC-goat anti-mouse IgC. Percentage of positive stained cells in each circle is indicated (Middle and Bottom) Percentage and means of fluorescence intensity of CD158⁺ and HLA-Cw3⁺ cells are indicated.

Similar results were obtained with splenocytes isolated from Tg CD158b L61 transgenic mice as compared with PBL. Of note, we also detected human KIR on the surface of mouse B cells. This result indicates that the cell surface expression of KIR does not require any T/NK-specific molecular environment, as previously demonstrated in COS fibroblasts as well as in the RBL-2H3 mast cell line.

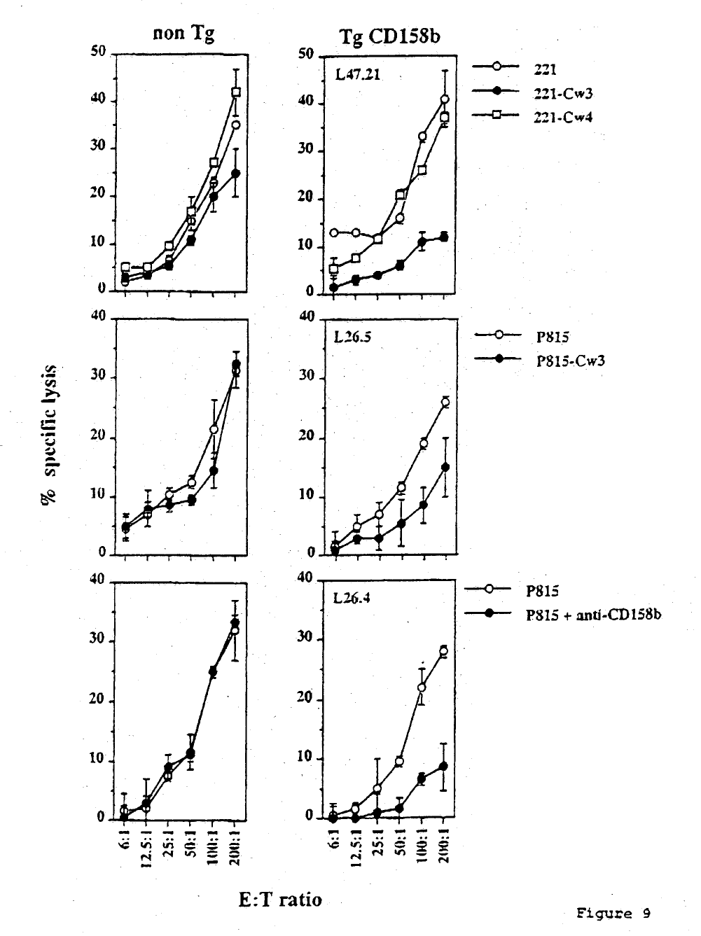

Splenocytes isolated from nontransgenic and CD158b transgenic mice were then analyzed for their ability to induce lysis of human HLA class I negative (221) and murine (P815) tumor cell lines transfected or not with HLA-Cw3. Results are reported on FIG. 9. FIG. 9 shows the in vitro cytotoxicity of splenic NK cells isolated from CD158b transgenic mice. Freshly isolated nonadherent splenocytes from CD158b transgenic (Tg CD158b) mice (L47 and L26 mouse lines) and nontransgenic littermate (non Tg) were tested for their ability to kill the indicated target cell lines in a standard 4-hr cytotoxicity assay. The following mice were used in this representative experiment: L47,21 (H-2^{k/b}), L26,4 (H-2^{b/b}), and L26,5 (H-^{k/b}).

Splenocytes isolated from the CD158b Tg mice were unable to induce an efficient lysis of both 221-Cw3 and P815-Cw3 cells. By contrast, HLA2-Cw3⁺ target cells were not protected from: lysis exerted by splenocytes isolated from the nontransgenic mice. Of note, splenocytes that express or not the CD158b transgene were able to induce lysis of 221, 221-Cw4, and P815 cell lines (FIG. 9 Top and Middle). Thus, the expression of HLA-Cw3 at the surface of target cell line selectively inhibits the natural cytotoxicity of splenocytes that express the CD158b transgene. Cross-linking of CD158b using anti-CD158b mAb mimicked the effect of HLA-Cw3 (FIG. 9 Bottom), and consistent with observations performed in human NK clones, KIR engagement is always more efficient with anti-KIR mAb than with the cognate MHC class I molecule.

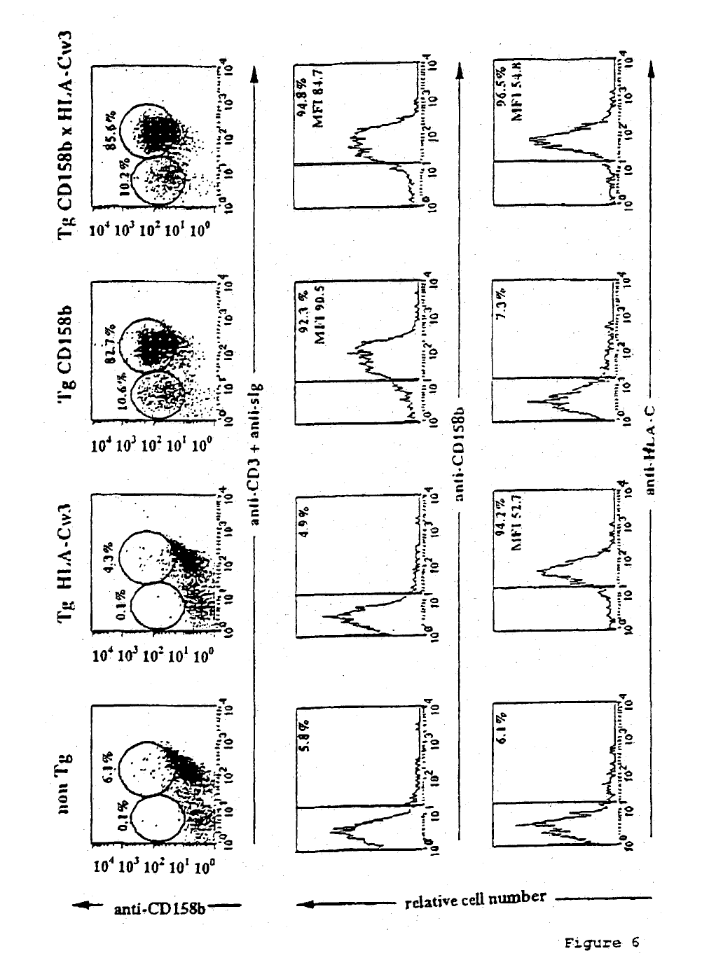

The function of the transgenic CD158b molecule expressed at the surface of mouse T cells was then analyzed. Results are reported on FIG 7. FIG. 7 shows the in vivo cytotoxicity of splenic T cells isolated from CD158b transgenic mice. Freshly isolated nonadherent splenocytes from CD158b transgenic mice (Tg CD158b, L26 mouse line) and nontransgenic littermates (non Tg) were tested in a redirected killing assay against P815 target cells at an effector; target ratio of 100:1, Anti-CD3 mAb-induced cytotoxicity was inhibited in Tg CD158b T cells upon CD158b engagement by HLA-Cw3 expressed on target cells (FIG. 7A) or by anti-CD158b mAb (FIG. 7B). Neither HLA-Cw3 expression (FIG. 7A) not anti-CD158b mAb (FIG. 7B) could induce inhibition of anti-CD3 redirected target cell lysis by non-Tg T cells. The following mice were used in this representative experiment: L26.4(H-2^{b/b}), and L26.5 (H-^{k/b}).

The CD3/T cell receptor complex was engaged using anti-CD3ε mAb in a redirected killing assay toward P815 cells. The engagement of CD158b by HLA-Cw3 (FIG. 7A) or by anti-CD158b mAb (FIG. 7B) inhibited the anti-CD3-mediated redirected killing of P815 by T cells from CD158b transgenic animals. The cell surface expression of HLA-Cw3 did not protect P815 cells from lysis by T cells isolated from nontransgenic littermates. Therefore, the transgenic expression of CD158b reconstitutes its inhibitory function on both T and NK cell activation programs in in vitro cytotoxicity assays.

CD158b expression is not infineaced by the expression of its HLA-Cw3 ligand in vivo. In an attempt to document the influence of the cognate MHC class I molecules on the cell surface expression of their KIR ligand, CD158b transgenic mince were crossed to mice transgenic for the CD158b ligand, HLA-Cw3. As shown in FIG. 6, no difference could be detected as to percentage of CD158b⁻ NK (CD3⁻, sIg⁻ cells) and CD158b⁺ T/B cells (CD3+, sIg⁺) between the CD158b single transgenic and the CD158b X HLA-Cw3 double transgenic mice. In addition, no modulation of CD158b cell surface expression could be observed either, as assessed by the mean fluorescence intensity of CD158b: CD158b mean fluorescence intensity was 84±10 and 78±8 in PBL isolated from CD158b transgenic and CD158b X HLA-Cw3 double transgenic mice, respectively (P>0.6). Similarly, the cell surface expression of HLA-Cw3 was unchanged between the single HLA-Cw3 transgenic mice when compared with the double CD158b X HLA-Cw3 transgenic mice (FIG. 6). Thus, in our experimental model, we canot detect any adaptation of KIR cell surface expression to that their HLA class I ligands.

Prevention of HLA-Cw3⁺, H-2 mismatched bone marrow graft rejection in CD158b transgenic mice. It has been previously demonstrated that NK cells from an irradiated H-2^{k/b} hybrid host mediate the rejection of mismatched H-2^{k/k} or H-2^{b/b} parental bone marrow grafts. The role of the CD158b KIR transgene was then tested in vivo for its ability to modulate the rejection of bone marrow graft in a similar hybrid resistance assay. Bone marrow grafts were prepared from HLA-Cw3 transgenic mice of H-2^{b/b} haplotype. Syngenic H-2^{b/b} HLA-Cw3 transgenic mice, H-2^{k/b} HLA-Cw3 transgenic mice, and H-2^{k/b} CD158b X HLA-Cw3 transgenic mice were used as hosts following lethal irradiation. The syngeric H-2^{b/b} HLA-Cw3 graft was successful, whereas the H-2^{k/b} HLA-Cw3 transgenic mice rejected H-2^{b/b} bone marrow grafts.

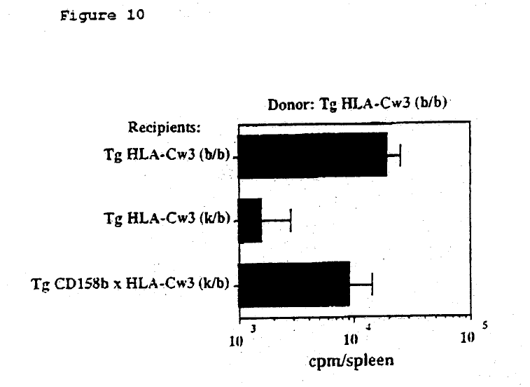

Results are reported on FIG. 10 which illustrates that CD158b transgenic mice are tolerant to graft of allogeneic bone marrow cells that express HLA-Cw3. Incorporation of ¹²⁵IdUdr in donor marrow-derived cells in the spleen of irradiated recipients 6 days after bone marrow graft was used as an assay to determine the extent of donor cell proliferation. Results are expressed as mean cpm=SEM of incorporated ¹²⁵IdUdr obtained from three independent grafts.

This result confirms the H-2^{k/b} hybrid resistance to H-2^{b/b} parental grafts as a consequence of the lack of expression of inhibitory receptors for H-2^{b} (potentially Ly-49C) on NK cell subsets from the H-2^{k/b} HLA-Cw3 transgenic mice. By contrast, H-2^{b/b} bone marrow grafts were not rejected in H-2^{k/b} CD158b X HLA-Cw3 transgenic mice despite the mismatch at the H-2 locus. Therefore, the engagement of the transgenic C158b KIR in hybrid host cells overcomes the lack of expression of endogenous KIRs, which recognize H-2^{b} molecules. Moreover, these results demonstrate that the inhibitory signals generated upon engagement of CD158b with its HLA-Cw3 ligand override the signals initiated by the endogenous mouse activatory receptors expressed on NK cells, similar to CD158b dominant inhibition of endogenous activatory receptors. Since it has been shown that human KIRs inhibitory function depends upon the recruitment of protein tyrosine phosphatases (i.e., SHP-1) by their intracytoplasmic immunoreceptor tyrosine-based inhibition motifs, our results are in agreement with data indicating that both human and mouse NK cell activatory receptors use a common protein tyrosine kinase-dependent signaling pathway.

### Discussion

The identification of KIRs revealed a novel strategy for T and NK, cell control that is based on the promiscuous recognition of MHC class I molecules on antigen-presenting cells and target cells. Human KIRs belong to two unrelated familes of molecules, IgSF (CD158b, p70, P410) or dimeric C-type lectins (CD94-NKG2A/B), whereas only dimeric C-type lectins KIRs (Ly-49) have been described in the mouse. In vitro experiments using anti-KIR mAbs as well as KIR gene transfection have shown that engagement of human IgSF KIRs with their MHC class I ligands inhibit both T and NK cell activation programs (see the herein above examples). In vivo experiments in unmanipulated as well as transgenic mice have shown that the absence of mouse lectin KIRs is responsible for the F₁ rejection of MHC class I mismatch parental bone marrow graft. By contrast, no data are available relative to the role of human IgSF KIRs in vivo. our data demonstrate that CD158b is sufficent to confer specificity to NK cells in vitro (FIGS. 9 and 7) and in vivo (FIG. 10). The generation of human IgSF KIR transgenic mice reported here also provides several answers to central issues on the function and the selection of human KIRs.

First, these results represent the first experimentals in vivo evidence that human IgSF KIRs control the host tolerance to MHC mismatch bone marrow grafts. In the hybrid resistance experimental system that we used, only NK cells from the hybrid F, are responsible for the rejection of parental bone marrow grafts (FIG. 10). The inhibition of anti-CD3-induced T cell cytotoxicity by KIR engagement (FIG. 7) enlarges the spectrum of KIR inhibitory function, and reveals that both T and NK cells from the CD158b transgenic mice are unresponsive to any activatory stimuli when HLA-Cw3 interacts with CD158b. Therefore, our results provide an explanation for the necessity of selecting for a KIR expression confined to NK and T cell subsets. Indeed, the expression of KIR reacting with self-MHC on all T cells would prevent their response to antigen. Moreover, the distribution of KIPs on all NK cells rather that on NK cell subsets, as it naturally occurs, would render these cells insensitive to changes in the expression of only one MHC class I allele, which is a frequent alteration of MHC class I expression observed in vivo upon viral infection or malignant transformation.

Second, it is of note that in the double CD:158b X HLA-Cw3 transgenic mice we cannot detect any adaptation of KIR cell surface expression to its MHC class I ligand (FIG. 6). This results is consistent with the lack of correlation between the level of expression of p70/NKBL as well as the frequency of p70/NKBL⁺ cells, and the expression of cognate MHC class I molecules (i.e., HLA-Bw4). In the mouse, a model of “receptor calibration” has been proposed based on the observation that the level of Ly-49 expression is down-regulated in the H-2 background corresponding to its ligand (e.g., H2-D² for Ly-49A). This adaptation of mouse KIR to their H-2 ligands selects for a low level of KIR cell surface expression and allows NK cells to detect subtle alteration of self-MHC class I expression. We can rule out the possibility that the use of an exogenous promoter for the generation of the CD158b transgenic mice might have influenced our observation, since a down-regulation of a Ly-49A transgene driven by the same promoter was detected in H-2^{d} mice. Therefore, the absence of adaptation of CD158b KIR cell surface expression to HLA-Cw3 in the double CD158b X HLA-Cw3 transgenic mice would rather suggest that distinct strategies of selection/calibration are used by human IgSF KIRs and mouse lectin-like KIRs. In this regard, our results also indicate that the interaction between IgSF KIRs and their cognate MHC class I ligands experts no role in the proliferation and differentiation of NK and T lymphocytes that express KIRs in contrast to the inhibition of their cytotoxic programs. It is therefore possible that KIRs are unable to inhibit cytokine-induced lymphocyte proliferation once it is initiated, but rather selectively impair the signaling cascades that drive the cell cycle from G₀ to G₁, such as antigen-induced T cell activation. We have described in the above examples, that the coligation between KIRs and various activatory receptors is mandatory to KIR inhibitory function. Consistent with this observation, two factors are likely to determine the efficiency of KIR inhibitory function: (i) the intensity of the activatory signals and (ii) the ratio between the number of KIRs and the number of activatory receptors coexpressed on the same cell. The transgenic expression of KIR is up-regulated in peripheral T cells as compared with immature thymocytes and mimics the up-regulation of human IgSF KIRs during their progressions from thymocytes to naive and memory T cells. The low expression of KIRs at early phases or T and NK cell development could thus account for their inability to inhibit T and NK cell differentiation. It remains also to be elucidated whether KIRs are coupled to an inhibitory signaling pathway (i.e., protein tyrosine phosphatases) only at a later stage of their differentiation programs and/or whether the signaling pathways that are coupled to the cytokine receptors involved in thymocytes/T cell and NK cell differentitation/proliferation are refractory to KIR inhibition.

Finally, it has been recently described that in patients receiving a haplo-identical bone marrow graft, a large fraction of the reconstituted T cell population expresses IgSF KIRs at their surface. Expression of KIRs may thus prevent the development of an immune response mounted against the cells of the host. Taken together with the acceptance of HLA-Cw3⁺ H-2 mismatched bone marrow grafts by CD158b transgenic mice reported here, these results emphasize the implications of documenting and acting on KIR expression in the development of novel strategies of cellular therapy.

### EXAMPLE 5

Preparation of a bispecific diantibody capable of cross-linking a KIR with a stimulatory receptor in the intracytoplasmic domain

It has been shown (see example 3, antiserum 712) that rabbits can be immunized using synthetic p58.2 ITIM peptides. In these experiments, the ITIM peptides were coupled to ovalbumin. This data thus demonstrates that one can obtain specific anti-ITIM antibodies.

Using a similar immunization strategy, monoclonal antibodies directed against the intracytoplasmic domain of several ITIM-bearing molecules, including phosphorylated and non-phosphorylated KIR ITIMs can be generated.

Similarly, antibodies can be generated against the intracytoplasmic domains of ITAM-polypeptides included in the CD3/TCR, FcεRI as well as FcγIIIA receptor complexes.

In parallel, soluble fusion protein corresponding to the extracytoplasmic domain of KIRs can serve as immunogens to generate antibodies.

Diantibodies can therefore be generated from the above-mentioned antibodies by standard procedure.

As an example of bispecific antibodies, mAbs directed against the ectodomain of p58.2 KIR (the inhibitory receptor for HLA-Cw3) can be chemically coupled to mAbs directed towards the ectodomain of CD3ζ. In these experiments, purified anti-p58.2 mAbs (GL183, mouse IgG1) and anti-CD3ε mAbs (mouse IgG) are obtained from Immunotech (Marseille, France). To GL183 mAbs (2-5 mg/ml in HBS) or their F(ab′)₂ fragments (obtained by pepsine digestion by standard procedure is added a 10-fold molar excess or EMCS (N-hydroxysuccinimidyl-6-maleimidocaproate, Fluka, Buchs, Switzerland; 10 mg/ml in methanol). The mixture is incubated for 1 hour at room temperature. Excess EMCS is removed by gel filtration on a PD-10 column (Pharmacia, Bois d'Arcy, France) presaturated with bovine serum albumine (BSA) and equilibrated in HBS-5 mM EDTA, pH 7.2. Anti-CD3 F(ab′)₂ fragments are reduced with cysteamine (10 mM, 1 hour, 37° C.) and mixed to EMCS-derivatized GL183 F(ab′)₂ fragments in a 1.5:1 molar ratio and allowed to react at room temperature for 24 hours. DSC (dual specificity conjugates) were separated from unreacted fragments by gel filtration on a TSK column (Pharmacia) in PBS-0.02% NaN₃. Fractions corresponding to an apparent molecular weight of 150000 [F(ab′)₂-Fab′ DSC] or 100000 [Fab′-Fab′ DSC] are collected, pooled, filtered through 0.22 μm filters (Amicon, Paris, France) and stored at 4° C. Control DSCs can be prepared as described above by coupling an anti-CD56 mAb (Immunotech) and GL183 mAbs. Separated products are identified by SDS-PAGE on an automated apparatus (PhastSystem), using 8-25% gradient PhastGels and coomassie blue staining (Pharmacia). All protein solutions are concentrated by positive pressure ultracentrifugation using PM-10 membranes (Amicon). Protein concentrations for IgC, fragments, and DSC are determined by absorbance at 280 nM (assuming 1.0 mg/ml=1.4 absorbance units). The p58.2-CD3 DSCs induce the co-aggregation between p58.2 KIR and the CD3/TCR complexes expressed on subpopulation of 58.2⁺ T cells in a dose-dependent manner. Incubation of sorted p58.2⁺ T cells with saturating concentrations of p58.2-CD3 DSC (50 μg/ml) for 40 minutes a 4° C. prevents anti-CD3-driven T cell activation induced by non competing anti-CD3 mAbs. Based on this protocol, DSCs made of a variety of mAbs directed towards ITAM- and ITIM-bearing receptors coexpressed at the surface of the same cells can be prepared (e.g. CD16 and KIRs on NK cells, BCR and FcγRIIBL on B cells, FcεRI and FcγRIIBL on mast cells and basophils), and will inhibit in vivo and in vitro cell activation induced by the engagement of the ITAM-bearing receptors. The mechanisms of action DSC are based on the signaling disruption exerted by ITIM-bearing receptors on ITAM-bearing receptors to which they are co-aggregated. A screening on the serotonin release of RBL-2H3 cell transfectants allows the selection of the most efficient compounds (diantibody, peptide, glycoprotein, carbohydrate).

1/ Compound capable of cross-linking a stimulatory receptor with a KIR.

2/ Compound according to claim 1, characterized in that it is capable of specifically regulating the activation of a KIR.

3/ Compound according to claim 1 or 2, characterized in that it is capable of regulating the activation of a stimulatory receptor.

4/ Compound according to any of the preceeding claims characterized in that said stimulatory receptor is an ITAM-bearing receptor such as KAR FcεRI, CD3/TCR, CD16, receptors related to tyrosine kinase activities or a receptor sub-unit such as CD3ζ, CD3ε, CD3γ, CD3δ or FcεRIγ.

5/ Compound according to any one of claims 1-4, characterized in that said KIR is a IgSF member, particularly selected from the group comprising CD158, CDw159, CDw160, or said KIR is lectin-like, such as the CD94-NKG2A/B heterodimer.

6/ Compound according to any of the preceeding claims, characterized in that said KIR is expressed on a NK cell, on a T cell, on a mast cell or on a monocyte or is recombinantly expressed.

7/ Compound according to any of the preceeding claims, characterized in that it is capable of inducing the regulation of free calcium concentration in a cell, particularly of inducing the regulation of calcium influx into a cell and/or of inducing the regulation of calcium mobilization from intracellular compartments.

8/ Compound according to any of the preceeding claims, characterized in that it is capable of inducing the recruitment by said KIR or KIR homologue of a phosphatase selected from the group consisting of SHP-1, SHP-2.

9/ Compound according to any of the preceeding claims, characterized in that it is essentially a polypeptide, a glycoprotein or a carbohydrate.

10/ Compound according to any of the preceeding claims, characterized in that said compound is a bispecific reagent and/or a chemical inducer of dimerization.

11/ Compound according to any of the preceeding claims, characterized in that said compound is a bispecific antibody, comprising at least one Fab, Fd, Fv, dAb, CDR, F(ab′)₂, VH, VL, ScFv fragment.

12/ Compound according to any of the preceeding claims, characterized in that it is capable of cross-linking said KIR with said stimulatory receptor in the extracellular domain of a cell.

13/ Compound according to any of the preceeding claims, characterized in that it is capable of crossing through a lipid bi-layer, and is liposoluble and/or associated with a drug-delivery system.

14/ Compound according to any of the preceeding claims, characterized in that it is capable of cross-linking said KIR with said stimulatory receptor in the intracellular domain of a cell.

15/ Compound according to any of the preceeding claims, characterized in that it is capable of modulating the release of serotonin and/or of inflammatory mediators by a cell expressing FcεRI, such as a mast cell, and/or of modulating cytokine release, such as Interleukin-6, Tumor Necrosis Factor Alpha release, from a cell such as a mast cell or a NK cell and/or of modulating interleukin production such as the IL-2 production and/or the γ-interferon production from a peripheral blood cell and/or of modulating the proliferation of peripheral blood cells.

16/ Compound according to any of the preceeding claims, characterized in that it is capable of controlling the host tolerance to allogeneic grafts and/or the graft toxicity against a host tissue.

17/ Nucleic acid coding for a polypeptide according to any one of claims 9-16.

18/ Cell transfected by a nucleic acid according to claim 17.

19/ Pharmaceutical preparation comprising a compound according to any one of claims 1-16 or a nucleic acid according to claim 17 or a cell according to claim 18 in a a physiologically acceptable vehicle, in a therapeutically-effective amount useful for modulating an animal cell function involved in a disease selected from the group consisting of immunoproliferative diseases, immunodeficiency diseases, cancers, autoimmune diseases, infectious diseases, viral diseases, inflammatory responses, allergic responses or involved in organ transplant tolerance.

20/ Method for the in vitro or ex vivo diagnosis of a cell disregulation, comprising the step of estimating of the relative proportion of co-aggregated KIR vs. non-co-aggregated KIR by:
contacting a biological sample with a compound according to any one of claims 1-16 or with a nucleic acid according to claim 17 or a cell according to claim 18, and
revealing the reaction product possibly formed.
